(19) United States
(12) Patent Application Publication
(10) Pub. No.: US 2023/0229082 A2
(43) Pub. Date: Jul. 20, 2023

(54) RADIATION-SENSITIVE RESIN COMPOSITION AND METHOD OF FORMING RESIST PATTERN

(71) Applicant: JSR CORPORATION, Tokyo (JP)

(72) Inventor: Katsuaki NISHIKORI, Tokyo (JP)

(73) Assignee: JSR CORPORATION, Tokyo (JP)

(21) Appl. No.: 17/543,789

(22) Filed: Dec. 7, 2021

Related U.S. Application Data
(63) Continuation of application No. PCT/JP2020/020186, filed on May 21, 2020.

(30) Foreign Application Priority Data
Jun. 14, 2019 (JP) 2019-111482

(51) Int. Cl. G03F 7/039; C08F 220/18; C07D 327/04; G03F 7/038; C07C 309/12; C07C 309/06; C07C 381/12; C07D 317/72; G03F 7/004

(52) U.S. Cl. CPC G03F 7/039; C08F 220/1808; C07D 327/04; G03F 7/038; C07C 309/12; C07C 309/06; C08F 220/1806; C07C 381/12; C07D 317/72; C08F 220/1812; C08F 220/1805; C08F 220/1807; C08F 220/1809; G03F 7/0045; C07C 2603/74

(57) ABSTRACT

A radiation-sensitive resin composition includes: a polymer which has a first structural unit including a phenolic hydroxyl group, and a second structural unit represented by formula (1); and a radiation-sensitive acid generating agent which has a compound represented by formula (2). R¹ represents a hydrogen atom, or the like; R² represents a hydrogen atom or the like; and R³ represents a divalent monocyclic alicyclic hydrocarbon group having 3 to 12 ring atoms. Ar¹ represents a group obtained by removing (q+1) hydrogen atoms on an aromatic ring from an arene formed by condensation of at least two benzene rings; R⁴ represents a monovalent organic group having 1 to 20 carbon atoms; q is an integer of 0 to 7; and R⁵ represents a halogen atom, a hydroxy group, a nitro group, or a monovalent organic group having 1 to 20 carbon atoms, or the like.

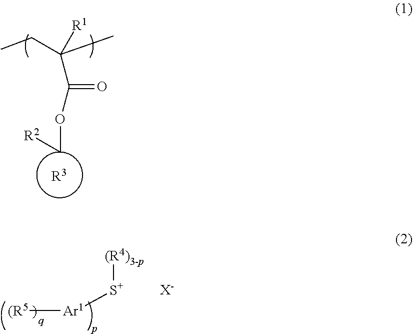

### CROSS-REFERENCE TO RELATED APPLICATIONS

The present application is a continuation application of International Application No. PCT/JP2020/020186, filed May 21, 2020, which claims priority to Japanese Patent Application No. 2019-111482 filed Jun. 14, 2019. The contents of these applications are incorporated herein by reference in their entirety.

### BACKGROUND OF THE INVENTION

### Field of the Invention

The present invention relates to a radiation-sensitive resin composition and a method of forming a resist pattern.

### Description of the Related Art

A radiation-sensitive resin composition for use in microfabrication by lithography generates an acid at a light-exposed region upon an irradiation with a radioactive ray, e.g., an electromagnetic wave such as a far ultraviolet ray such as an ArF excimer laser beam (wavelength of 193 nm), a KrF excimer laser beam (wavelength of 248 nm), etc., an extreme ultraviolet ray (EUV) (wavelength of 13.5 nm), or a charged particle ray such as an electron beam. A chemical reaction in which the acid serves as a catalyst causes a difference in rates of dissolution in a developer solution between light-exposed regions and light-unexposed regions, whereby a resist pattern is formed on a substrate.

Such a radiation-sensitive resin composition is required not only to have favorable sensitivity to exposure light such as an extreme ultraviolet ray and an electron beam, but also to have superiority with regard to LWR (Line Width Roughness) performance, which indicates line width uniformity, and resolution.

Types, molecular structures, and the like of polymers, acid generating agents, and other components which may be used in radiation-sensitive resin compositions have been investigated to meet these requirements, and combinations thereof have been further investigated in detail (see Japanese Unexamined Patent Applications, Publication Nos. 2016-040598 and 2007-206638).

### SUMMARY OF THE INVENTION

According to an aspect of the present invention, a radiation-sensitive resin composition includes: a polymer which includes: a first structural unit including a phenolic hydroxyl group; and a second structural unit represented by formula (1); and a radiation-sensitive acid generating agent which includes a compound represented by formula (2).

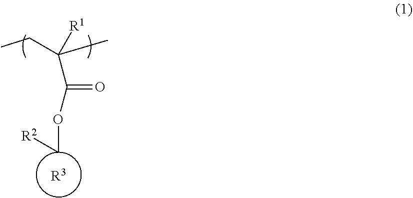

In the formula (1), R¹ represents a hydrogen atom, a fluorine atom, a methyl group, or a trifluoromethyl group; R² represents a hydrogen atom or a monovalent hydrocarbon group having 1 to 20 carbon atoms; and R³ represents a divalent monocyclic alicyclic hydrocarbon group having 3 to 12 ring atoms.

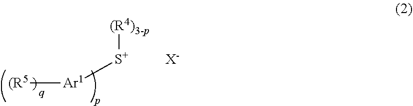

In the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two of the plurality of R⁵s taken together represent an alicyclic structure having 4 to 20 ring atoms or an aliphatic heterocyclic structure having 4 to 20 ring atoms, together with the carbon chain to which the at least two of the plurality of R⁵s bond; in a case in which p is no less than 2, a plurality of Ar¹s are identical or different from each other, and a plurality of q's are identical or different from each other; and X⁻ represents a monovalent anion.

According to another aspect of the present invention, a radiation-sensitive resin composition includes: a polymer which includes a first structural unit represented by formula (5), and a second structural unit represented by formula (6); and a radiation-sensitive acid generating agent which includes a compound represented by formula (2).

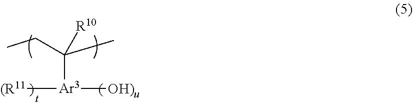

In the formula (5), R¹⁰ represents a hydrogen atom, a fluorine atom, a methyl group, or a trifluoromethyl group; Ar³ represents a group obtained by removing (t+u+1) hydrogen atoms on an aromatic ring from an arene having 6 to 20 ring atoms; t is an integer of 0 to 10, wherein in a case in which t is 1, R¹¹ represents a halogen atom or a monovalent organic group having 1 to 20 carbon atoms, and in a case in which t is no less than 2, a plurality of R¹¹s are identical or different and each R¹¹ represents a halogen atom or a monovalent organic group having 1 to 20 carbon atoms, or at least two of the plurality of R¹¹s taken together represent a ring structure having 4 to 20 ring atoms together with the carbon chain to which the at least two of the plurality of R¹¹s bond; and u is an integer of 1 to 11, wherein a sum of t and u is no greater than 11.

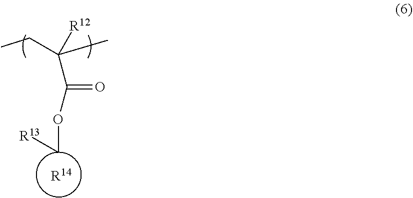

In the formula (6), R¹² represents a hydrogen atom, a fluorine atom, a methyl group, or a trifluoromethyl group; R¹³ represents a hydrogen atom or a monovalent hydrocarbon group having 1 to 20 carbon atoms; and R¹⁴ represents a divalent alicyclic hydrocarbon group having 3 to 30 ring atoms.

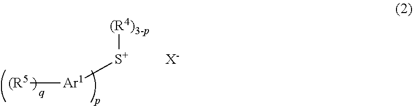

In the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two R⁵s taken together represent an alicyclic structure having 4 to 20 ring atoms or an aliphatic heterocyclic structure having 4 to 20 ring atoms, together with the carbon chain to which the at least two R⁵s bond; in a case in which p is no less than 2, a plurality of Ar¹s are identical or different from each other, and a plurality of q's are identical or different from each other; and X⁻ represents a monovalent anion.

According to a further aspect of the present invention, a method of forming a resist pattern includes forming a resist film directly or indirectly on a substrate by applying the above-mentioned radiation-sensitive resin composition. The resist film is exposed. The resist film exposed is developed.

### DESCRIPTION OF EMBODIMENTS

Under current circumstances in which miniaturization of resist patterns has proceeded to a level in which line widths are 40 nm or less, required levels for the aforementioned types of performance are further elevated.

According to one embodiment of the invention, a radiation-sensitive resin composition (hereinafter, may be also referred to as “composition (I)”) contains: a polymer (hereinafter, may be also referred to as “(A1) polymer” or “polymer (A1)”) which has a first structural unit including a phenolic hydroxyl group, and a second structural unit represented by the following formula (1); and a radiation-sensitive acid generating agent (hereinafter, may be also referred to as “(B) acid generating agent” or “acid generating agent (B)”) which includes a compound represented by the following formula (2):

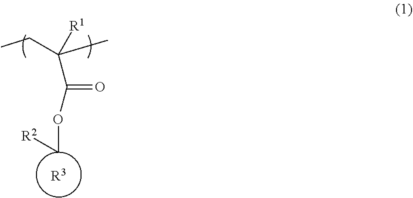

wherein, in the formula (1), R¹ represents a hydrogen atom, a fluorine atom, a methyl group, or a trifluoromethyl group; R² represents a hydrogen atom or a monovalent hydrocarbon group having 1 to 20 carbon atoms; and R³ represents a divalent monocyclic alicyclic hydrocarbon group having 3 to 12 ring atoms, and

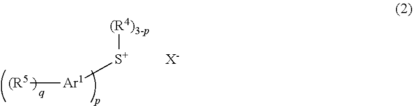

in the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two of the plurality of R⁵s taken together represent an alicyclic structure having 4 to 20 ring atoms or an aliphatic heterocyclic structure having 4 to 20 ring atoms, together with the carbon chain to which the at least two of the plurality of R⁵s bond; in a case in which p is no less than 2, a plurality of Ar¹s are identical or different from each other, and a plurality of q's are identical or different from each other; and X⁻ represents a monovalent anion.

According to another embodiment of the invention, a radiation-sensitive resin composition (hereinafter, may be also referred to as “composition (II)”) contains: a polymer (hereinafter, may be also referred to as “(A2) polymer” or “polymer (A2)”) which has a first structural unit represented by the following formula (5), and a second structural unit represented by the following formula (6); and a radiation-sensitive acid generating agent (the acid generating agent (B)) which includes a compound represented by the following formula (2):

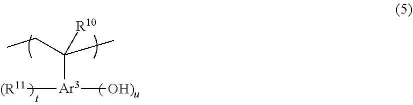

wherein, in the formula (5), R¹⁰ represents a hydrogen atom, a fluorine atom, a methyl group, or a trifluoromethyl group; Ar³ represents a group obtained by removing (t+u+1) hydrogen atoms on an aromatic ring from an arene having 6 to 20 ring atoms; t is an integer of 0 to 10, wherein in a case in which t is 1, R¹¹ represents a halogen atom or a monovalent organic group having 1 to 20 carbon atoms, and in a case in which t is no less than 2, a plurality of R¹¹s are identical or different and each R¹¹ represents a halogen atom or a monovalent organic group having 1 to 20 carbon atoms, or at least two of the plurality of R¹¹s taken together represent a ring structure having 4 to 20 ring atoms together with the carbon chain to which the at least two of the plurality of R¹¹s bond; and u is an integer of 1 to 11, wherein a sum of t and u is no greater than 11,

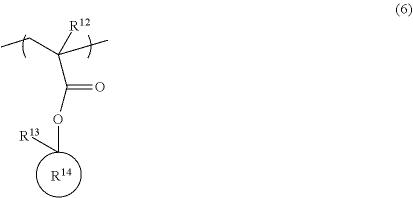

in the formula (6), R¹² represents a hydrogen atom, a fluorine atom, a methyl group, or a trifluoromethyl group; R¹³ represents a hydrogen atom or a monovalent hydrocarbon group having 1 to 20 carbon atoms; and R¹⁴ represents a divalent alicyclic hydrocarbon group having 3 to 30 ring atoms, and

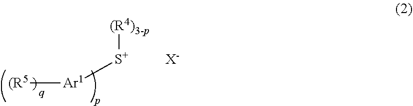

in the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two R⁵s taken together represent an alicyclic structure having 4 to 20 ring atoms or an aliphatic heterocyclic structure having 4 to 20 ring atoms, together with the carbon chain to which the at least two R⁵s bond; in a case in which p is no less than 2, a plurality of Ar¹s are identical or different from each other, and a plurality of q's are identical or different from each other; and X⁻ represents a monovalent anion.

According to still another embodiment of the invention, a method of forming a resist pattern includes: applying the radiation-sensitive resin composition of the one embodiment of the present invention directly or indirectly on a substrate; exposing a resist film formed by the applying; and developing the resist film exposed.

The radiation-sensitive resin composition and the method of forming a resist pattern of the embodiments of the present invention enable a resist pattern to be formed with favorable sensitivity to exposure light and superiority with regard to each of the LWR performance and the resolution. Therefore, these can be suitably used in manufacturing processes of semiconductor devices, in which further progress of miniaturization is expected in the future. Hereinafter, the embodiments of the present invention will be explained in detail.

### Radiation-Sensitive Resin Composition

Modes of the radiation-sensitive resin composition include the composition (I) and the composition (II) shown below.

Composition (I): contains the polymer (A1) and the acid generating agent (B).

Composition (II): contains the polymer (A2) and the acid generating agent (B).

It is to be noted that herein, the polymer (A1) and the polymer (A2) may be collectively referred to as the “polymer (A).”

The radiation-sensitive resin composition may be used either for positive-tone pattern formation conducted using an alkaline developer solution, or for negative-tone pattern formation conducted using an organic solvent-containing developer solution.

The radiation-sensitive resin composition is for exposure by irradiation with a radioactive ray (exposure light) in the exposing in the method of forming a resist pattern, to be described later. Of types of exposure light, an extreme ultraviolet ray (EUV) or an electron beam has comparatively high energy, but even in the case of using an extreme ultraviolet ray or an electron beam as the exposure light, the radiation-sensitive resin composition enables a resist pattern to be formed with favorable sensitivity to the exposure light and superiority with regard to the LWR performance and resolution. Accordingly, the radiation-sensitive resin composition can be particularly suitably used for exposure with an extreme ultraviolet ray or exposure with an electron beam.

Hereinafter, with regard to the radiation-sensitive resin composition, the composition (I) and the composition (I) will be explained, in this order.

Composition (I)

The composition (I) contains the polymer (A1) and the acid generating agent (B). The composition (I) may contain, as favorable component(s), an acid diffusion controller (hereinafter, may be also referred to as “acid diffusion controller (C)”) and/or an organic solvent (hereinafter, may be also referred to as “organic solvent (D)”), and may also contain, within a range not leading to impairment of the effects of the present invention, other optional component(s).

Due to containing the polymer (A1) and the acid generating agent (B), the composition (I) has favorable sensitivity to exposure light, and enables a resist pattern to be formed with superiority with regard to each of the LWR performance and the resolution. Although not necessarily clarified and without wishing to be bound by any theory, the reason for achieving the aforementioned effects by the composition (I) due to involving such a constitution may be presumed, for example, as in the following. Due to the acid generating agent (B) contained in the composition (I) having a sulfonium cation having a specific structure represented by the above formula (2), an amount of the acid generated increases. It is considered that as a result, combining the polymer (A1) and the acid generating agent (B) enables a resist pattern to be formed with favorable sensitivity to exposure light and superiority with regard to each of the LWR performance and the resolution.

Each component contained in the composition (I) is described below.

(A1) Polymer

The polymer (A1) has the first structural unit (hereinafter, may be also referred to as “structural unit (I-1)”) including a phenolic hydroxyl group, and the second structural unit (hereinafter, may be also referred to as “structural unit (I-2)”) represented by the following formula (1). The polymer (A1) may also have an other structural unit aside from the structural unit (I-1), and the structural unit (I-2). The polymer (A1) may have one, or two or more types of each structural unit.

Each structural unit contained in the polymer (A1) will be described below.

Structural Unit (I-1)

The structural unit (I-1) contains a phenolic hydroxyl group. The “phenolic hydroxyl group” as referred to herein is not limited to a hydroxy group directly bonding to a benzene ring, and means any hydroxy group directly bonding to an aromatic ring in general. When the polymer (A1) contains the structural unit (I-1), hydrophilicity of the resist film can be increased, solubility in a developer solution can be appropriately adjusted, and further, adhesiveness of the resist pattern to the substrate can be improved. Furthermore, in a case of using an extreme ultraviolet ray or an electron beam as the radioactive ray for irradiation in a step of irradiating of the method of forming a resist pattern, as described later, the sensitivity to exposure light can be further improved.

Examples of the structural unit (I-1) include structural units represented by the following formula (4), and the like.

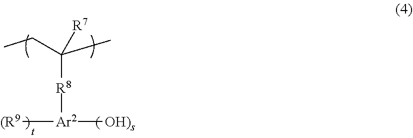

In the above formula (4), R⁷ represents a hydrogen atom, a fluorine atom, a methyl group, or a trifluoromethyl group; R⁸ represents a single bond, —O—, —COO—, or —CONH—; Ar² represents a group obtained by removing (r+s+1) hydrogen atoms on an aromatic ring from an arene having 6 to 20 ring atoms; r is an integer of 0 to 10, wherein in a case in which r is 1, R⁹ represents a halogen atom or a monovalent organic group having 1 to 20 carbon atoms, and in a case in which r is no less than 2, a plurality of R⁹s are identical or different and each R⁹ represents a halogen atom or a monovalent organic group having 1 to 20 carbon atoms, or at least two of the plurality of R⁹s taken together represent a ring structure having 4 to 20 ring atoms together with the carbon chain to which the at least two of the plurality of R⁹s bond; and s is an integer of 1 to 11, wherein a sum of r and s is no greater than 11.

In light of copolymerizability of a monomer that gives the structural unit (I-1), R⁷ represents preferably a hydrogen atom or a methyl group.

In the case in which R⁸ represents —COO—, the oxy-oxygen atom is preferably bonded to Ar², and in the case in which R⁸ represents —CONH—, the nitrogen atom is preferably bonded to Ar². More specifically, in the case in which * denotes a binding site to Ar², —COO— is preferably —COO—*, and —CONH— is preferably —CONH—*. R⁸ represents preferably a single bond or —COO—, and more preferably a single bond.

The number of “ring atoms” as referred to herein means the number of atoms constituting the ring in an alicyclic structure, an aromatic ring structure, an aliphatic heterocyclic structure, or an aromatic heterocyclic structure, and in the case of a polycyclic ring structure, the number of “ring atoms” means the number of atoms constituting the polycyclic ring.

Examples of the arene having 6 to 20 ring atoms that gives Ar² include benzene, naphthalene, anthracene, phenanthrene, tetracene, pyrene, and the like. The arene having 6 to 20 ring atoms that gives Ar² is preferably benzene or naphthalene, and more preferably benzene.

The “organic group” as referred to herein means a group that includes at least one carbon atom. The number of “carbon atoms” as referred to herein means the number of carbon atoms constituting a group. The monovalent organic group having 1 to 20 carbon atoms which may be represented by R⁹ is exemplified by: a monovalent hydrocarbon group having 1 to 20 carbon atoms; a group (α) that includes a divalent hetero atom-containing group between two adjacent carbon atoms of the monovalent hydrocarbon group having 1 to 20 atoms; a group (β) obtained by substituting with a monovalent hetero atom-containing group, a part or all of hydrogen atoms included in the monovalent hydrocarbon group having 1 to 20 atoms or the group (α); a group (γ) obtained by combining the monovalent hydrocarbon group having 1 to 20 atoms, the group (α), or the group (β) with a divalent hetero atom-containing group; and the like.

The “hydrocarbon group” as referred to herein may be exemplified by a chain hydrocarbon group, an alicyclic hydrocarbon group, and an aromatic hydrocarbon group. The “hydrocarbon group” may be either a saturated hydrocarbon group or an unsaturated hydrocarbon group. The “chain hydrocarbon group” as referred to herein means a hydrocarbon group not including a cyclic structure but being constituted with only a chain structure, and may be exemplified by both a linear hydrocarbon group and a branched hydrocarbon group. The “alicyclic hydrocarbon group” as referred to herein means a hydrocarbon group that includes, as a ring structure, not an aromatic ring structure but an alicyclic structure alone, and may be exemplified by both a monocyclic alicyclic hydrocarbon group and a polycyclic alicyclic hydrocarbon group. With regard to this, it is not necessary for the alicyclic hydrocarbon group to be constituted with only an alicyclic structure; it may include a chain structure in a part thereof. The “aromatic hydrocarbon group” as referred to herein means a hydrocarbon group that includes an aromatic ring structure as a ring structure. With regard to this, it is not necessary for the aromatic hydrocarbon group to be constituted with only an aromatic ring structure; it may include a chain structure or an alicyclic structure in a part thereof.

The monovalent hydrocarbon group having 1 to 20 carbon atoms is exemplified by a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent chain hydrocarbon group having 1 to 20 carbon atoms include: alkyl groups such as a methyl group, an ethyl group, an n-propyl group, and an i-propyl group; alkenyl groups such as an ethenyl group, a propenyl group, and a butenyl group; alkynyl groups such as an ethynyl group, a propynyl group, and a butynyl group; and the like.

Examples of the monovalent alicyclic hydrocarbon group having 3 to 20 carbon atoms include: alicyclic saturated hydrocarbon groups such as a cyclopentyl group, a cyclohexyl group, a norbornyl group, an adamantyl group, a tricyclodecyl group, and a tetracyclododecyl group; alicyclic unsaturated hydrocarbon groups such as a cyclopentenyl group, a cyclohexenyl group, a norbornenyl group, a tricyclodecenyl group, and a tetracyclododecenyl group; and the like.

Examples of the monovalent aromatic hydrocarbon group having 6 to 20 carbon atoms include: aryl groups such as a phenyl group, a tolyl group, a xylyl group, a naphthyl group, and an anthryl group; aralkyl groups such as a benzyl group, a phenethyl group, a naphthylmethyl group, and an anthrylmethyl group; and the like.

The hetero atom constituting the monovalent hetero atom-containing group or the divalent atom-containing group is exemplified by an oxygen atom, a nitrogen atom, a sulfur atom, a phosphorus atom, a silicon atom, and a halogen atom. Examples of the halogen atom include a fluorine atom, a chlorine atom, a bromine atom, an iodine atom, and the like.

Examples of the divalent hetero atom-containing group include —O—, —CO—, —S—, —CS—, —NR′—, a combination of two or more of these, and the like. R′ represents a hydrogen atom or a monovalent hydrocarbon group.

R⁹ represents preferably the monovalent hydrocarbon group having 1 to 20 carbon atoms, and more preferably the alkyl group.

Examples of the ring structure having 4 to 20 ring atoms represented by the at least two of the plurality of R⁹s taken together, together with the carbon atom to which the at least two of the plurality of R⁹s bond, include alicyclic structures such as a cyclopentane structure, a cyclohexane structure, a cyclopentene structure, a cyclohexene structure, and the like.

r is preferably 0 to 2, more preferably 0 or 1, and still more preferably 0.

s is preferably 1 to 3, and more preferably 1 or 2.

Examples of the structural unit (I-1) include structural units (hereinafter, may be also referred to as “structural units (I-1-1) to (I-1-12)”) represented by the following formulae (4-1) to (4-12), and the like.

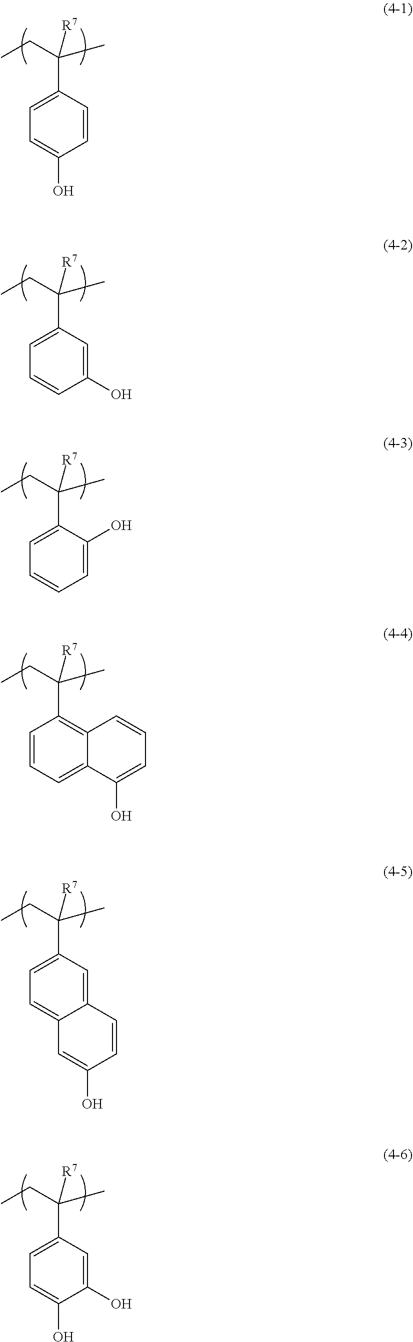

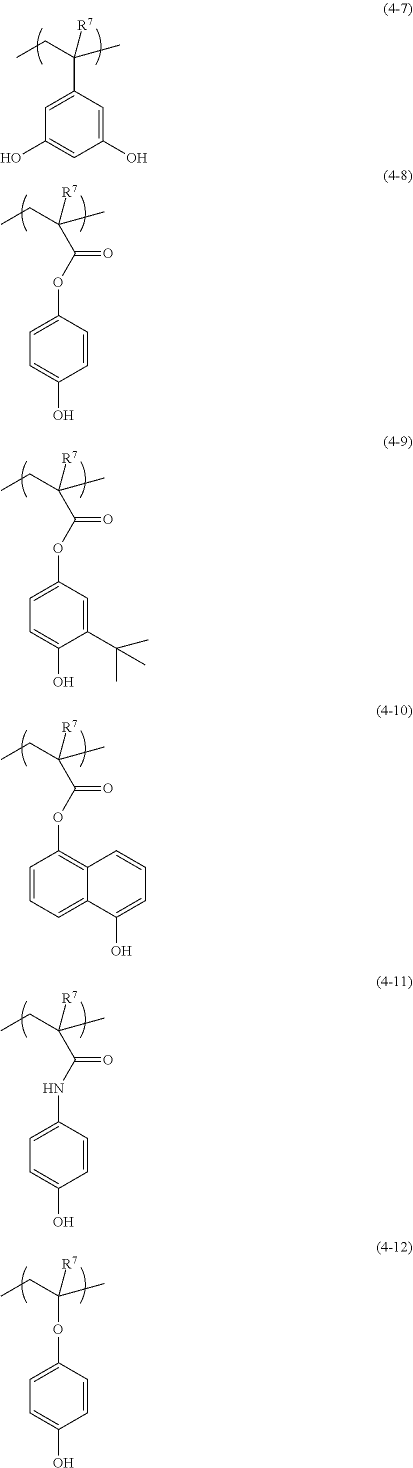

In the above formulae (4-1) to (4-12), R⁷ is as defined in the above formula (4).

The structural unit (I-1) is preferably the structural unit (I-1-1) or the structural unit (I-1-2).

The lower limit of a proportion of the structural unit (I-1) in the polymer (A1) contained with respect to total structural units constituting the polymer (A1) is preferably 20 mol %, more preferably 25 mol %, and still more preferably 30 mol %. The upper limit of the proportion is preferably 60 mol %, more preferably 55 mol %, and still more preferably 50 mol %. When the proportion of the structural unit (I-1) falls within the above range, the sensitivity to exposure light of the composition (I), as well as the LWR performance and the resolution of the resist pattern formed therefrom can be further improved.

Structural Unit (I-2)

The structural unit (I-2) is represented by the following formula (1). The structural unit (I-2) includes an acid-labile group. The “acid-labile group” as referred to herein means a group that substitutes for a hydrogen atom of a carboxy group, and is dissociable by an action of an acid. When the polymer (A1) contains the acid-labile group in the structural unit (I-2), the acid-labile group is dissociated in light-exposed regions by an action of an acid generated from the acid generating agent (B) in the exposing, and a difference in solubility in a developer solution emerges between the light-exposed regions and the light-unexposed regions, thereby enabling forming the resist pattern. It is to be noted that in the following formula (1), a group bonding to an oxy-oxygen atom derived from the carboxy group corresponds to the acid-labile group.

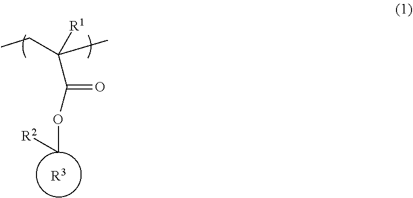

In the above formula (1), R¹ represents a hydrogen atom, a fluorine atom, a methyl group, or a trifluoromethyl group; R² represents a hydrogen atom or a monovalent hydrocarbon group having 1 to 20 carbon atoms; and R³ represents a divalent monocyclic alicyclic hydrocarbon group having 3 to 12 ring atoms.

In light of copolymerizability of a monomer that gives the structural unit (I-2), R¹ represents preferably a hydrogen atom or a methyl group.

Examples of the monovalent hydrocarbon group having 1 to 20 carbon atoms which may be represented by R² include hydrocarbon groups similar to those exemplified as R⁹ in the above formula (4), and the like. It is to be noted that as represented in the above formula (1), R² corresponds to a group bonding to the carbon atom in R³ which bonds to the oxy-oxygen atom.

Examples of the divalent monocyclic alicyclic hydrocarbon group having 3 to 12 ring atoms represented by R³ include: groups obtained by removing two hydrogen atoms from one carbon atom constituting a monocyclic saturated alicyclic structure such as a cyclopentane ring or a cyclohexane ring; groups obtained by removing two hydrogen atoms from one carbon atom constituting a monocyclic unsaturated alicyclic structure such as a cyclopentene ring or a cyclohexene ring; and the like.

The monovalent hydrocarbon group having 1 to 20 carbon atoms which may be represented by R² is preferably a hydrocarbon group other than a polycyclic alicyclic hydrocarbon group, and more preferably a monovalent chain hydrocarbon group having 1 to 20 carbon atoms or a monovalent aromatic hydrocarbon group having 6 to 20 carbon atoms.

In the case in which R² represents a hydrogen atom, R³ represents preferably a monocyclic alicyclic unsaturated hydrocarbon group.

In the case in which R² represents the monovalent hydrocarbon group having 1 to 20 carbon atoms, R³ represents preferably a monocyclic alicyclic saturated hydrocarbon group.

Examples of the structural unit (I-2) include structural units (hereinafter, may be also referred to as “structural units (I-2-1) to (I-2-7)”) represented by the following formulae (1-1) to (1-7), and the like.

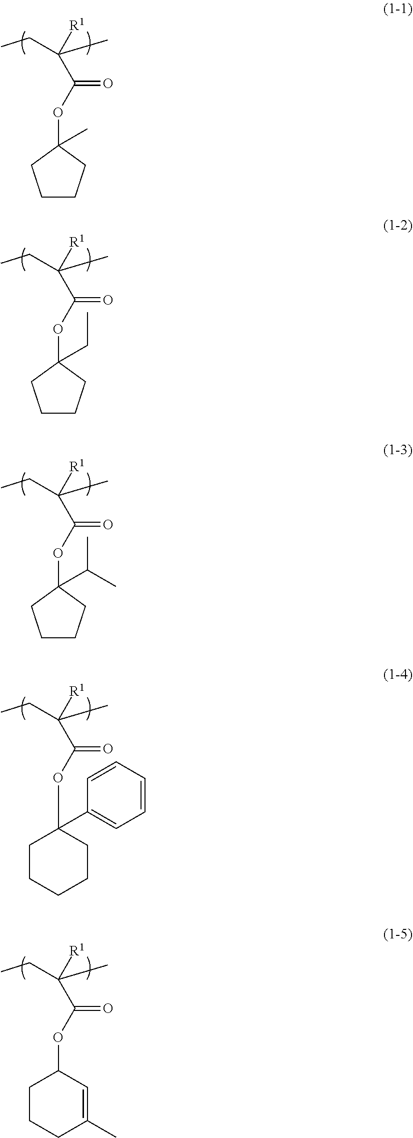

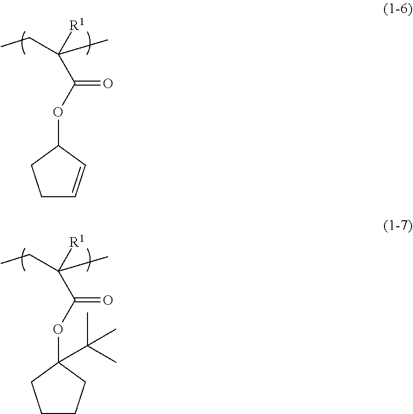

In the above formulae (1-1) to (1-7), R¹ is as defined in the above formula (1).

The lower limit of a proportion of the structural unit (I-2) in the polymer (A1) contained with respect to total structural units constituting the polymer (A1) is preferably 20 mol %, more preferably 30 mol %, and still more preferably 40 mol %. The upper limit of the proportion is preferably 90 mol %, more preferably 80 mol %, and still more preferably 70 mol %. When the proportion of the structural unit (I-2) falls within the above range, the sensitivity to exposure light of the composition (I), as well as the LWR performance and the resolution of the resist pattern formed therefrom can be further improved.

Other Structural Unit

The other structural unit may be exemplified by a structural unit (hereinafter, may be also referred to as “structural unit (I-3)”) including an alcoholic hydroxyl group; a structural unit (hereinafter, may be also referred to as “structural unit (I-4)”) including a lactone structure, a cyclic carbonate structure, a sultone structure, or a combination thereof; and the like. When the polymer (A1) further has the structural unit (I-3), the structural unit (I-4), or a combination thereof, solubility in a developer solution can be even more appropriately adjusted, and as a result, the sensitivity to exposure light of the composition (I) and the LWR performance of the resist pattern formed therefrom can be even further improved. Furthermore, adhesiveness of the resist pattern to the substrate can be even further improved.

Structural Unit (I-3)

Examples of the structural unit (I-3) include structural units represented by the following formulae, and the like.

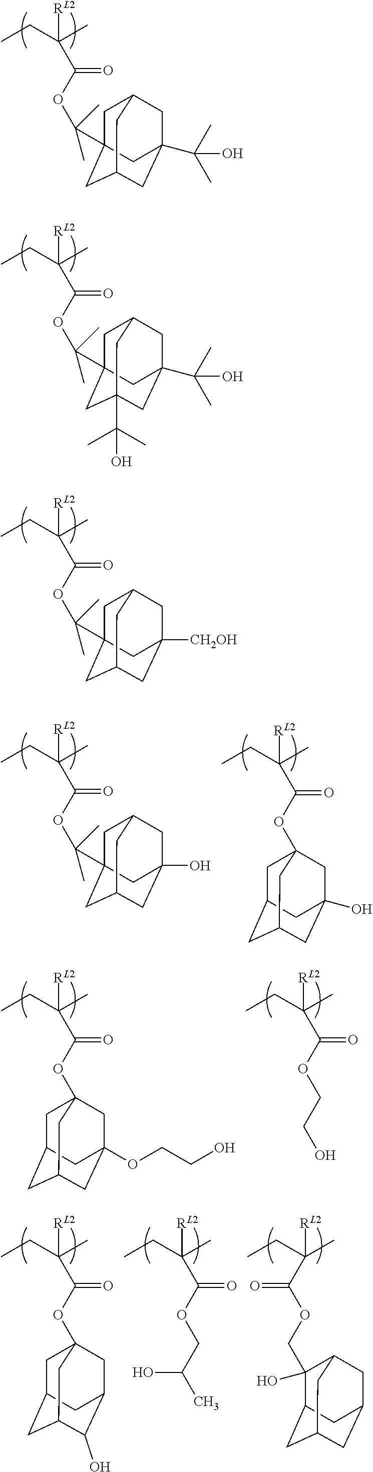

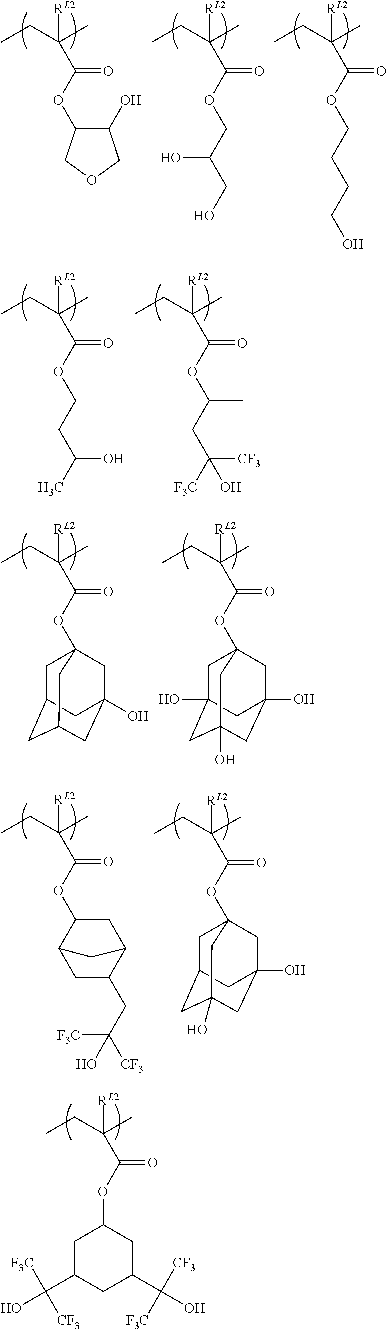

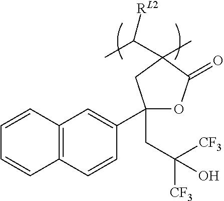

In each of the above formulae, R^{L2} represents a hydrogen atom, a fluorine atom, a methyl group, or a trifluoromethyl group.

In the case in which the polymer (A1) has the structural unit (I-3), the lower limit of a proportion of the structural unit (I-3) contained with respect to total structural units in the polymer (A1) is preferably 1 mol % and more preferably 5 mol %. The upper limit of the proportion is preferably 20 mol %, and more preferably 15 mol %.

Structural Unit (I-4)

Examples of the structural unit (I-4) include structural units represented by the following formulae, and the like.

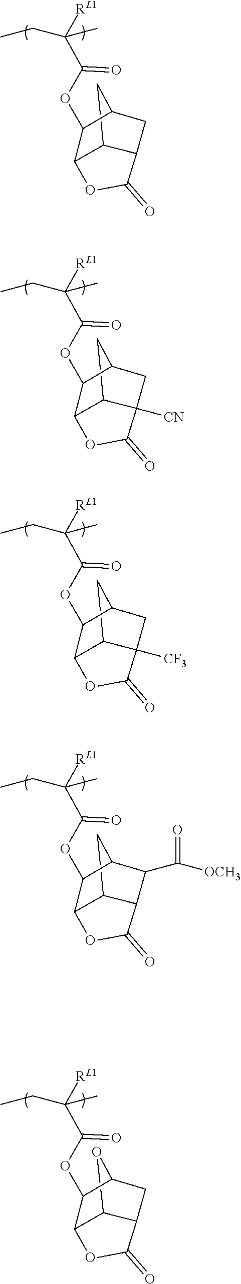

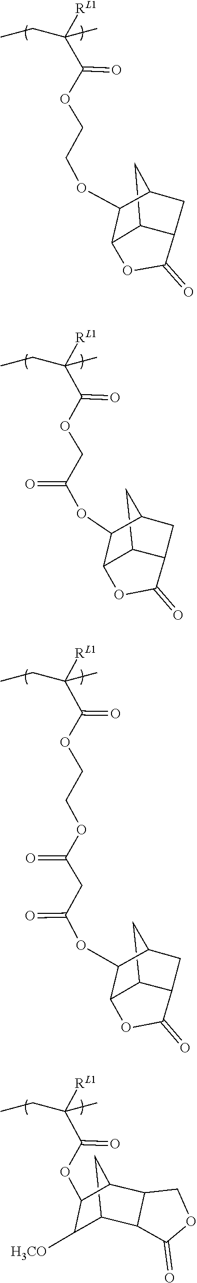

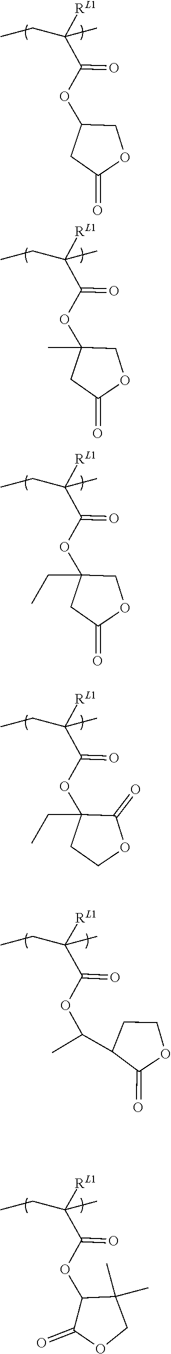

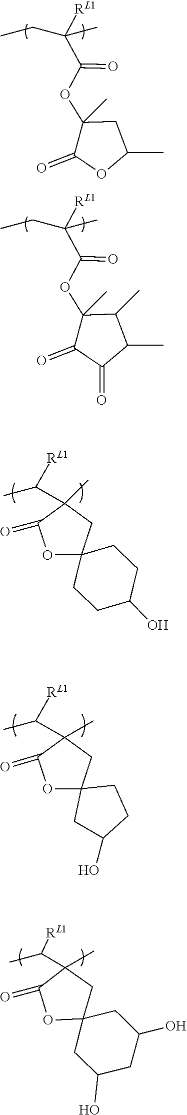

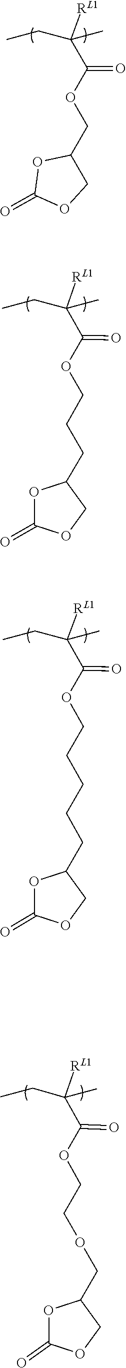

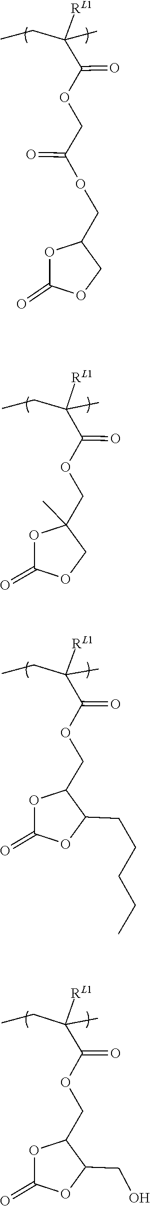

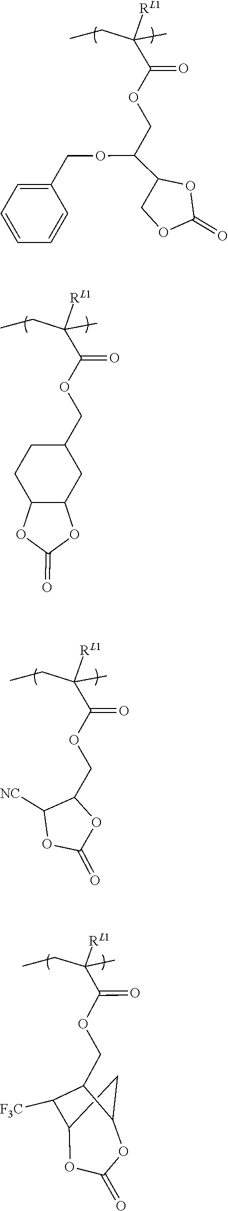

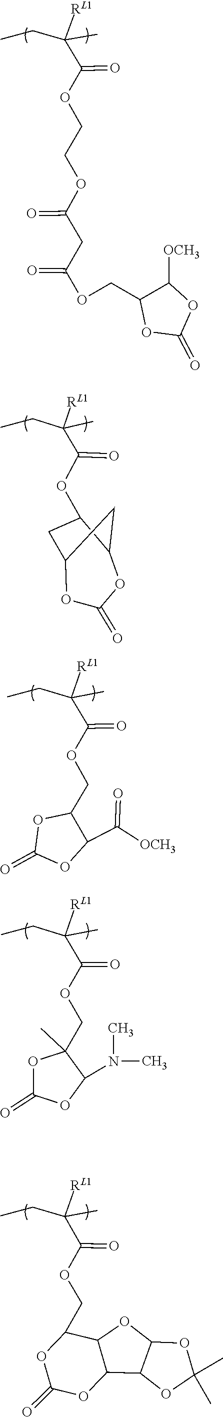

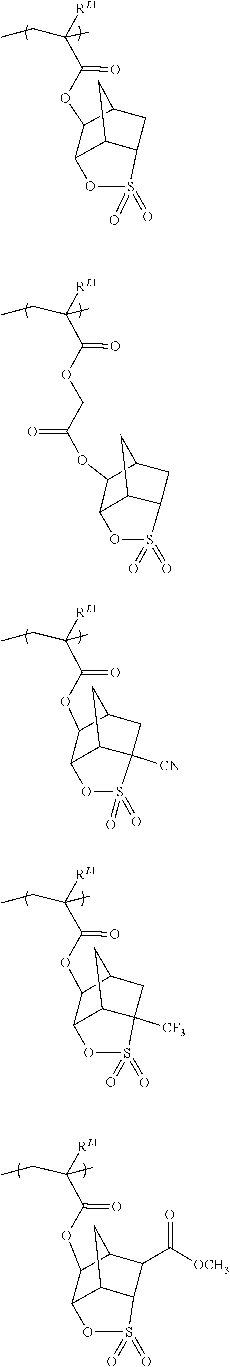

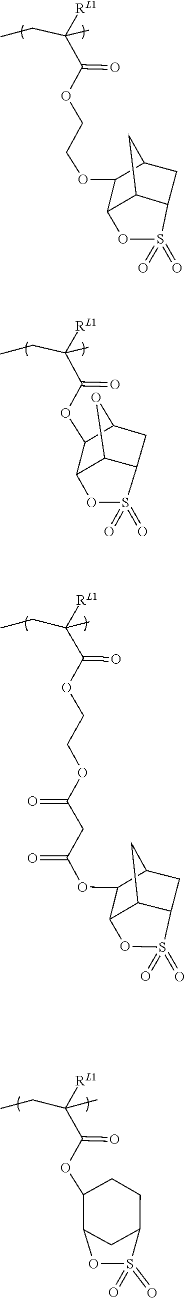

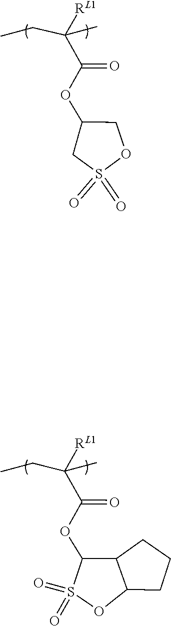

In each of the above formulae, R^{L1} represents a hydrogen atom, a fluorine atom, a methyl group, or a trifluoromethyl group.

The structural unit (I-4) preferably includes the lactone structure.

In the case in which the polymer (A1) has the structural unit (I-4), the lower limit of a proportion of the structural unit (I-4) contained with respect to total structural units in the polymer (A1) is preferably 1 mol % and more preferably 5 mol %. The upper limit of the proportion is preferably 30 mol %, and more preferably 20 mol %.

The lower limit of a polystyrene equivalent weight average molecular weight (Mw) of the polymer (A1) as determined by gel permeation chromatography (GPC) is preferably 2,000, more preferably 3,000, and still more preferably 4,000. The upper limit of the Mw is preferably 10,000, more preferably 9,000, and still more preferably 8,000. When the Mw of the polymer (A1) falls within the above range, solubility in a developer solution can be appropriately adjusted.

The upper limit of a ratio (Mw/Mn) of the Mw with respect to a polystyrene-equivalent number average molecular weight (Mn) of the polymer (A1) as determined by GPC is preferably 2.50, more preferably 2.00, and still more preferably 1.75. The lower limit of the ratio is typically 1.00, preferably 1.10, and more preferably 1.20. When the Mw/Mn of the polymer (A1) falls within the above range, coating characteristics of the composition (I) can be further improved.

As referred to herein, the Mw and Mn of the polymer are values determined by gel permeation chromatography (GPC) under the following conditions.

GPC columns “G2000 HXL”×2, “G3000 HXL”×1, and “G4000 HXL”×1, available from Tosoh Corporation;

elution solvent: tetrahydrofuran

flow rate: 1.0 mL/min

sample concentration: 1.0% by mass

amount of injected sample: 100 uL

column temperature: 40° C.

detector: differential refractometer

standard substance: mono-dispersed polystyrene

The lower limit of a proportion of the polymer (A1) in the composition (I) with respect to total components other than the organic solvent (D) is preferably 50% by mass, more preferably 60% by mass, still more preferably 70% by mass, and particularly preferably 80% by mass.

The polymer (A1) can be synthesized by, for example, polymerizing a monomer that gives each structural unit according to a well-known procedure.

(B) Acid Generating Agent

The acid generating agent (B) has a compound (hereinafter, may be also referred to as “compound (B)”) represented by the following formula (2). The compound (B) is a substance which generates an acid by irradiation with a radioactive ray. Examples of the radioactive ray include electromagnetic waves such as visible light rays, ultraviolet rays, far ultraviolet rays, extreme ultraviolet rays (EUV), X-rays, and γ-rays; charged particle rays such as electron beams and α-rays, and the like. An acid-labile group included in the structural unit (I-2) of the polymer (A1) is dissociated by an action of an acid generated from the compound (B) upon irradiation (exposure) with the radioactive ray, thereby generating a carboxy group and creating a difference in solubility in the developer solution of the polymer (A1) between a light-exposed region and a light-unexposed region: accordingly, a resist pattern can be formed.

The lower limit of a temperature at which the acid dissociates the acid-labile group is preferably 80° C., more preferably 90° C., and still more preferably 100° C. The upper limit of the temperature is preferably 130° C., more preferably 120° C., and still more preferably 110° C. The lower limit of a time period for the acid to dissociate the acid-labile group is preferably 10 sec, and more preferably 1 min. The upper limit of the time period is preferably 10 min, and more preferably 2 min.

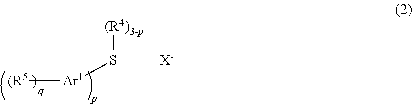

In the above formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two R⁵s taken together represent an alicyclic structure having 4 to 20 ring atoms or an aliphatic heterocyclic structure having 4 to 20 ring atoms together with the carbon chain to which the at least two R⁵s bond; in a case in which p is no less than 2, a plurality of Ar¹s are identical or different, and a plurality of q's are identical or different; and X⁻ represents a monovalent anion.

Examples of the arene formed by condensation of at least two benzene rings which gives Ar¹ include: naphthalene, anthracene, phenanthrene, phenalene, tetracene, triphenylene, pyrene, chrysene, picene, perylene, pentaphene, pentacene, hexaphene, hexacene, corenene, and the like. Of these, an arene formed by condensation of 2 to 4 benzene rings is preferred, an arene formed by condensation of 2 or 3 benzene rings is more preferred, and an arene formed by condensation of 2 benzene rings (specifically, naphthalene) is still more preferred.

Specifically, in the case in which the arene formed by condensation of at least two benzene rings which gives Ar¹ is naphthalene, the sulfur atom in the above formula (2) preferably bonds to a β-position of the naphthalene. It is to be noted that the β-position of naphthalene as referred to herein means position 2, 3, 6, or 7 of the naphthalene ring. When the sulfur atom bonds to the β-position of naphthalene, the resolution of the resist pattern formed by the composition (I) can be even further improved.

Examples of the monovalent organic group having 1 to 20 carbon atoms which may be represented by R⁴ or R⁵ include groups similar to the organic groups exemplified as R⁹ in the above formula (4), and the like.

R⁴ represents preferably a monovalent unsubstituted hydrocarbon group having 1 to 20 carbon atoms, or a hydrocarbon group obtained therefrom by substituting a part or all of hydrogen atoms contained therein with a substituent; more preferably a monovalent unsubstituted aromatic hydrocarbon group having 6 to 18 carbon atoms, or an aromatic hydrocarbon group obtained therefrom by substituting a part or all of hydrogen atoms contained therein with a substituent; still more preferably a substituted or unsubstituted phenyl group; and particularly preferably an unsubstituted phenyl group.

The substituent which may substitute for the hydrogen atom contained in the monovalent hydrocarbon group having 1 to 20 carbon atoms which may be represented by R⁴ is preferably —OSO₂—R^{k}, —SO₂—R^{k}, —OR^{k}, —COOR^{k}, —O—CO—R^{k}, —O—R^{k2}—COOR^{k}, —R^{k2}—CO—R^{k}, —S—R^{k}, or a substituted or unsubstituted monovalent hydrocarbon group having 1 to 20 carbon atoms, wherein R^{k} represents a monovalent hydrocarbon group having 1 to 10 carbon atoms, and R^{k2} represents a single bond or a divalent hydrocarbon group having 1 to 10 carbon atoms.

In the case in which q is 1, R⁵ represents preferably a hydroxy group or a group similar to the groups exemplified as the substituents which may substitute for the hydrogen atom contained in the monovalent hydrocarbon group having 1 to 20 carbon atoms represented by R⁴, and more preferably a hydroxy group.

In the case in which q is no less than 2 and the at least two of the plurality of R⁵s taken together represent an alicyclic structure having 4 to 20 ring atoms or an aliphatic heterocyclic structure having 4 to 20 ring atoms together with the carbon chain to which the at least two of the plurality of R⁵s bond, examples of these ring structures include: alicyclic structures such as a cyclopentane structure, a cyclohexane structure, a cyclopentene structure, and a cyclohexene structure; an oxygen atom-containing aliphatic heterocyclic structure; a nitrogen atom-containing aliphatic heterocyclic structure; a sulfur atom-containing aliphatic heterocyclic structure; and the like.

p is preferably 1 or 2, and more preferably 1. When p falls within the above range, solubility of the composition (I) in a developer solution can be further improved, and as a result, the sensitivity to exposure light of the composition (I), as well as the LWR performance and the resolution of the resist pattern formed therefrom can be even further improved.

q is preferably 0 to 2, more preferably 0 or 1, and still more preferably 0.

Examples of X⁻ include a monovalent anion (hereinafter, may be also referred to as “anion (X)”) represented by the following formula (3), and the like.

R⁶—Y⁻ (3)

In the above formula (3), R⁶ represents a monovalent organic group having 1 to 30 carbon atoms; and Y⁻ represents a group obtained by removing one proton from an acid group.

Examples of R⁶ include groups similar to the organic groups exemplified as R⁹ in the above formula (4), and the like.

The acid group that gives Y⁻ is exemplified by a sulfo group, a carboxy group, and the like. The acid group that gives Y⁻ is preferably the sulfo group. It is to be noted that in the case in which the acid group that gives Y⁻ is the sulfo group, X⁻ is a monovalent sulfonic acid anion, and in the case in which the acid group that gives Y⁻ is the carboxy group, X⁻ is a monovalent carboxylic acid anion.

Examples of the monovalent sulfonic acid anion include a sulfonic acid anion (hereinafter, may be also referred to as “anion (X-1)”) represented by the following formula (3′), and the like.

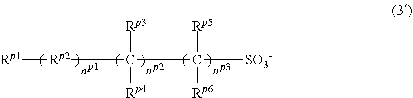

In the above formula (3′), R^{p1} represents a monovalent group including a ring structure having five or more ring atoms; R^{p2} represents a divalent linking group; R^{p3} and R^{p4} each independently represent a hydrogen atom, a fluorine atom, a monovalent hydrocarbon group having 1 to 20 carbon atoms, or a monovalent fluorinated hydrocarbon group having 1 to 20 carbon atoms; RP⁵ and RP⁶ each independently represent a fluorine atom or a monovalent fluorinated hydrocarbon group having 1 to 20 carbon atoms; n^{p1} is an integer of 0 to 10, n^{p2} is an integer of 0 to 10, and n^{p3} is an integer of 0 to 10, wherein a sum of n^{p1}, n^{p2}, and n^{p3} is no less than 1 and no greater than 30, in a case in which n^{p1} is no less than 2, a plurality of R^{p2}s are identical or different from each other, in a case in which n^{p2} is no less than 2, a plurality of R^{p3}s are identical or different from each other and a plurality of R^{p4}s are identical or different from each other, and in a case in which n^{p3} is no less than 2, a plurality of R^{p5}s are identical or different from each other and a plurality of R^{p6}s are identical or different from each other.

The monovalent group including a ring structure having five or more ring atoms represented by R^{p1} is exemplified by a monovalent group including an alicyclic structure having five or more ring atoms, a monovalent group including an aliphatic heterocyclic structure having five or more ring atoms, a monovalent group including an aromatic carbon ring structure having five or more ring atoms, a monovalent group including an aromatic heterocyclic structure having five or more ring atoms, and the like.

Examples of the alicyclic structure having five or more ring atoms include:

monocyclic saturated alicyclic structures such as a cyclopentane structure, a cyclohexane structure, a cycloheptane structure, a cyclooctane structure, a cyclononane structure, a cyclodecane structure, and a cyclododecane structure;

monocyclic unsaturated alicyclic structures such as a cyclopentene structure, a cyclohexene structure, a cycloheptene structure, a cyclooctene structure, and a cyclodecene structure;

polycyclic saturated alicyclic structures such as a norbornane structure, an adamantane structure, a tricyclodecane structure, and a tetracyclododecane structure;

polycyclic unsaturated alicyclic structures such as a norbornene structure and a tricyclodecene structure; and the like.

Examples of the aliphatic heterocyclic structure having five or more ring atoms include:

lactone structures such as a hexanolactone structure and a norbornanelactone structure;

sultone structures such as a hexanosultone structure and a norbornanesultone structure;

oxygen atom-containing heterocyclic structures such as an oxacycloheptane structure and an oxanorbornane structure;

nitrogen atom-containing heterocyclic structures such as an azacyclohexane structure and a diazabicyclooctane structure;

sulfur atom-containing heterocyclic structures such as a thiacyclohexane structure and a thianorbornane structure; and the like.

Examples of the aromatic carbon ring structure having five or more ring atoms include a benzene structure, a naphthalene structure, a phenanthrene structure, an anthracene structure, and the like.

Examples of the aromatic heterocyclic structure having five or more ring atoms include:

oxygen atom-containing heterocyclic structures such as a furan structure, a pyran structure, a benzofuran structure, and a benzopyran structure;

nitrogen atom-containing heterocyclic structures such as a pyridine structure, a pyrimidine structure, and an indole structure; and the like.

The lower limit of the number of ring atoms of the ring structure in R^{p1} is preferably 6, more preferably 8, still more preferably 9, and particularly preferably 10. The upper limit of the ring atoms is preferably 15, more preferably 14, still more preferably 13, and particularly preferably 12. When number of ring atoms falls within the above range, a diffusion length of the acid can be more appropriately shortened, and as a result, the sensitivity to exposure light of the radiation-sensitive resin composition and the LWR performance of the resist pattern formed therefrom can be further improved, and a process window can be further expanded.

A part or all of hydrogen atoms contained in the ring structure of R^{p1} may be substituted with a substituent. Examples of the substituent include halogen atoms such as a fluorine atom, a chlorine atom, a bromine atom, and an iodine atom; a hydroxy group; a carboxy group; a cyano group; a nitro group; an alkoxy group; an alkoxycarbonyl group; an alkoxycarbonyloxy group; an acyl group; an acyloxy group; and the like. Of these, a hydroxy group is preferred.

R^{p1} represents preferably a monovalent group including an alicyclic structure having five or more ring atoms, or a monovalent group including an aliphatic heterocyclic structure having five or more ring atoms; and more preferably a monovalent group including a polycyclic saturated alicyclic structure, a monovalent group including an oxygen atom-containing heterocyclic structure, or a monovalent group including a sulfur atom-containing heterocyclic structure.

Examples of the divalent linking group represented by R^{p2} include a carbonyl group, an ether group, a carbonyloxy group, a sulfide group, a thiocarbonyl group, a sulfonyl group, a divalent hydrocarbon group, and the like. Of these, the carbonyloxy group, the sulfonyl group, an alkanediyl group, or a divalent alicyclic saturated hydrocarbon group is preferred, and the carbonyloxy group is more preferred.

The monovalent hydrocarbon group having 1 to 20 carbon atoms which may be represented by R^{p3} or R^{p4} is exemplified by an alkyl group having 1 to 20 carbon atoms, and the like. The monovalent fluorinated hydrocarbon group having 1 to 20 carbon atoms which may be represented by R^{p3} or R^{p4} is exemplified by a fluorinated alkyl group having 1 to 20 carbon atoms, and the like. R^{p3} and R^{p4} each independently represent: preferably a hydrogen atom, a fluorine atom, or a fluorinated alkyl group; more preferably a fluorine atom or a perfluoroalkyl group; and still more preferably a fluorine atom or a trifluoromethyl group.

The monovalent fluorinated hydrocarbon group having 1 to 20 carbon atoms which may be represented by R^{p5} or R^{p6} is exemplified by a fluorinated alkyl group having 1 to 20 carbon atoms, and the like. R^{p5} and R^{p6} each independently represent preferably a fluorine atom or a fluorinated alkyl group, more preferably a fluorine atom or a perfluoroalkyl group, still more preferably a fluorine atom or a trifluoromethyl group, and particularly preferably a fluorine atom.

n^{p1} is preferably 0 to 5, more preferably 0 to 2, and still more preferably 0 or 1.

n^{p2} is preferably 0 to 5, more preferably 0 to 2, and still more preferably 0 or 1.

The lower limit of n^{p3} is preferably 1, and more preferably 2. When n^{p3} is no less than 1, strength of the acid can be enhanced, and as a result, the sensitivity to exposure light of the composition (I), as well as the LWR performance and the resolution of the resist pattern formed therefrom can be further improved. The upper limit of n^{p3} is preferably 4, more preferably 3, and still more preferably 2.

The lower limit of the sum of n^{p1}, n^{p2}, and n^{p3} is preferably 2, and more preferably 4. The upper limit of the sum of n^{p1}, n^{p2}, and n^{p3} is preferably 20, and more preferably 10.

Examples of the anion (X-1) include anions (hereinafter, may be also referred to as “anions (X-1-1) to (X-1-7)”) represented by the following formulae (3′-1) to (3′-7), and the like.

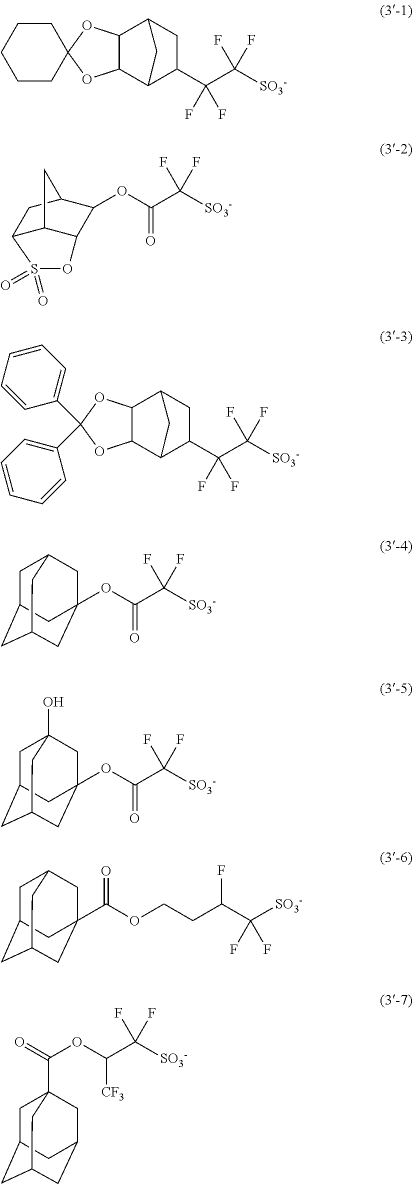

Examples of the compound (B) include compounds (hereinafter, may be also referred to as “compounds (B-1) to (B-6)”) represented by the following formulae (2-1) to (2-6).

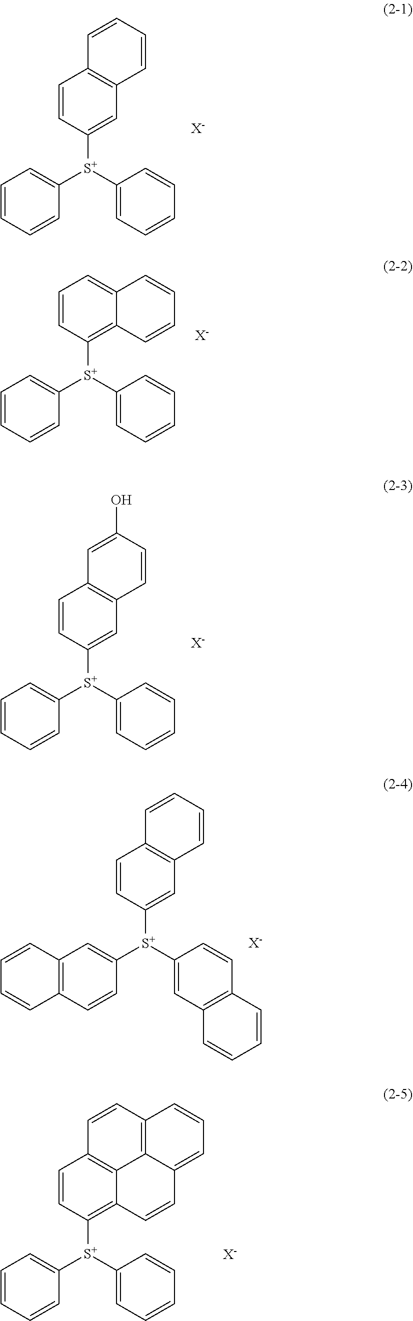

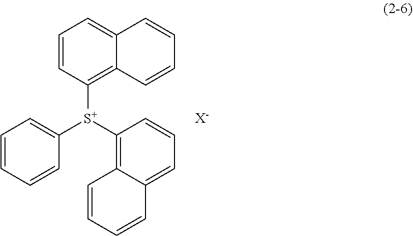

In the above formulae (2-1) to (2-6), X⁻ is as defined in the above formula (2).

The compound (B) is preferably the compound (2-1), (2-2), (2-3), (2-4), or (2-6), more preferably the compound (2-1), (2-2), (2-3), or (2-6), still more preferably the compound (2-1) or (2-2), and particularly preferably the compound (2-1).

The lower limit of a content of the acid generating agent (B) in the composition (I) with respect to 100 parts by mass of the polymer (A1) is preferably 0.1 parts by mass, more preferably 1 part by mass, and still more preferably 5 parts by mass. The upper limit of the content is preferably 70 parts by mass, more preferably 50 parts by mass, still more preferably 40 parts by mass, and particularly preferably 30 parts by mass. When the content of the acid generating agent (B) falls within the above range, the sensitivity to exposure light of the composition (I), as well as the LWR performance and the resolution of the resist pattern formed therefrom can be even further improved.

(C) Acid Diffusion Controller

The acid diffusion controller (C) is able to control a diffusion phenomenon in the resist film of the acid generated from the acid generating agent (B) and/or the like upon exposure, thereby serving to inhibit unwanted chemical reactions in a light-unexposed region. Furthermore, improving storage stability of the composition (I) and further improving the resolution are enabled. Moreover, changes in line width of the pattern caused by variation of post-exposure time delay from the exposure until a development treatment can be suppressed, thereby enabling the radiation-sensitive resin composition to be obtained having superior process stability. The acid diffusion controller (C) may be contained in the composition (I) in the form of a low-molecular-weight compound (hereinafter, may be referred to as “(C) acid diffusion control agent” or “acid diffusion control agent (C)” as appropriate) or in the form of an acid generator incorporated as a part of a polymer such as the polymer (A1), or may be in a combination of both these forms.

The acid diffusion control agent (C) is exemplified by a nitrogen atom-containing compound, a photodegradable base that is photosensitized by exposure to generate a weak acid, and the like.

Examples of the nitrogen atom-containing compound include: amine compounds such as tripentylamine and trioctylamine; amide group-containing compounds such as formamide and N,N-dimethylacetamide; urea compounds such as urea and 1,1-dimethylurea; nitrogen-containing heterocyclic compounds such as pyridine, N-(undecylcarbonyloxyethyl)morpholine, and N-t-pentyloxycarbonyl-4-hydroxypiperidine; and the like.

The photodegradable base is exemplified by a compound containing an onium cation degraded by exposure, and an anion of a weak acid; and the like. In a light-exposed region, the photodegradable base generates a weak acid from: a proton generated upon degradation of the onium cation; and the anion of the weak acid, whereby acid diffusion controllability decreases.

Examples of the photodegradable base include compounds represented by the following formulae.

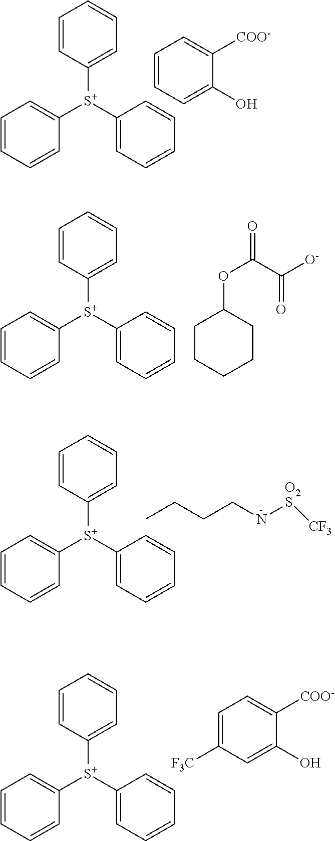

In the case in which the composition (I) contains the acid diffusion control agent (C), the lower limit of a content of the acid diffusion control agent (C) with respect to 100 parts by mass of the polymer (A1) is preferably 0.1 parts by mass, more preferably 0.5 parts by mass, and still more preferably 1 part by mass. The upper limit of the content is preferably 20 parts by mass, more preferably 10 parts by mass, and still more preferably 5 parts by mass.

In the case in which the composition (I) contains the acid diffusion control agent (C), the lower limit of a proportion of the acid diffusion control agent (C) with respect to 100 mol % of the acid generating agent (B) is preferably 1 mol %, more preferably 5 mol %, and still more preferably 10 mol %. The upper limit of the content is preferably 200 mol %, more preferably 100 mol %, and still more preferably 50 mol %.

When the content and/or the proportion of the acid diffusion control agent (C) fall(s) within the above range, the sensitivity to exposure light of the composition (I), as well as the LWR performance and the resolution of the resist pattern formed therefrom can be further improved. Either one, or two or more types of the acid diffusion controller (C) may be used.

Organic Solvent (D)

The composition (I) typically contains the organic solvent (D). The organic solvent (D) is not particularly limited as long as it is a solvent capable of dissolving or dispersing at least the polymer (A1) and the acid generator (B), as well as the optional component(s) which is/are contained as desired.

The organic solvent (D) is exemplified by an alcohol solvent, an ether solvent, a ketone solvent, an amide solvent, an ester solvent, a hydrocarbon solvent, and the like.

Examples of the alcohol solvent include:

aliphatic monohydric alcohol solvents having 1 to 18 carbon atoms such as 4-methyl-2-pentanol and n-hexanol;

alicyclic monohydric alcohol solvents having 3 to 18 carbon atoms such as cyclohexanol;

polyhydric alcohol solvents having 2 to 18 carbon atoms such as 1,2-propylene glycol;

polyhydric alcohol partial ether solvents having 3 to 19 carbon atoms such as propylene glycol-l-monomethyl ether; and the like.

Examples of the ether solvent include:

dialkyl ether solvents such as diethyl ether, dipropyl ether, dibutyl ether, dipentyl ether, diisoamyl ether, dihexyl ether, and diheptyl ether;

cyclic ether solvents such as tetrahydrofuran and tetrahydropyran;

aromatic ring-containing ether solvents such as diphenyl ether and anisole; and the like.

Examples of the ketone solvent include:

chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone;

cyclic ketone solvents such as cyclopentanone, cyclohexanone, cycloheptanone, cyclooctanone, and methylcyclohexanone;

2,4-pentanedione, acetonylacetone, and acetophenone; and the like.

Examples of the amide solvent include:

cyclic amide solvents such as N,N′-dimethylimidazolidinone and N-methylpyrrolidone;

chain amide solvents such as N-methylformamide, N,N-dimethylformamide, N,N-diethylformamide, acetamide, N-methylacetamide, N,N-dimethylacetamide, and N-methylpropionamide; and the like.

Examples of the ester solvent include:

monocarboxylic acid ester solvents such as n-butyl acetate and ethyl lactate;

lactone solvents such as γ-butyrolactone and valerolactone;

polyhydric alcohol carboxylate solvents such as propylene glycol acetate;

polyhydric alcohol partial ether carboxylate solvents such as propylene glycol monomethyl ether acetate;

polyhydric carboxylic acid diester solvents such as diethyl oxalate;

carbonate solvents such as dimethyl carbonate and diethyl carbonate; and the like.

Examples of the hydrocarbon solvent include:

aliphatic hydrocarbon solvents having 5 to 12 carbon atoms such as n-pentane and n-hexane;

aromatic hydrocarbon solvents having 6 to 16 carbon atoms such as toluene and xylene; and the like.

The organic solvent (D) is preferably the alcohol solvent or the ester solvent, more preferably the polyhydric alcohol partial ether solvent having 3 to 19 carbon atoms or the polyhydric alcohol partial ether carboxylate solvent, and still more preferably propylene glycol-1-monomethyl ether or propylene glycol monomethyl ether acetate. One, or two or more types of the organic solvent (D) may be contained.

In the case of the organic solvent (D) being contained in the radiation-sensitive resin composition, the lower limit of a proportion of the organic solvent (D) with respect to total components contained in the radiation-sensitive resin composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

Other Optional Component(s)

The other optional component(s) is/are exemplified by a surfactant and the like. The composition (I) may contain one, or two or more types each of the other optional component(s).

Surfactant

The surfactant achieves the effect of improving the coating characteristics, striation, developability, and the like. Examples of the surfactant include nonionic surfactants such as polyoxyethylene lauryl ether, polyoxyethylene stearyl ether, polyoxyethylene oleyl ether, polyoxyethylene n-octyl phenyl ether, polyoxyethylene n-nonyl phenyl ether, polyethylene glycol dilaurate, and polyethylene glycol distearate; and the like. Examples of a commercially available product of the surfactant include “KP341” (available from Shin-Etsu Chemical Co., Ltd.), “Polyflow No. 75” and “Polyflow No. 95” (each available from Kyoeisha Chemical Co., Ltd.), “EFTOP EF301,” “EFTOP EF303,” and “EFTOP EF352” (each available from JEMCO, Inc.), “MEGAFACE F171” and “MEGAFACE F173” (each available from Dainippon Ink and Chemicals, Inc.), “Fluorad FC430” and “Fluorad FC431” (each available from Sumitomo 3M Ltd.), “Asahi Guard AG710,” “Surflon S-382,” “Surflon SC-101,” “Surflon SC-102,” “Surflon SC-103.” “Surflon SC-104,” “Surflon SC-105,” and “Surflon SC-106” (each available from Asahi Glass Co., Ltd.), and the like.

In the case of the surfactant being contained in the composition (I), the upper limit of a content of the surfactant in the composition (I) with respect to 100 mol % of the polymer (A) is preferably 2 parts by mass. The lower limit of the content is, for example, 0.1 parts by mass.

Composition (II)

The composition (II) contains the polymer (A2) and the acid generating agent (B). Similarly to the composition (I), described above, the composition (II) may contain, as favorable component(s), the acid diffusion controller (C) and/or the organic solvent (D), and may also contain, within a range not leading to impairment of the effects of the present invention, the other optional component(s).

Due to containing the polymer (A2) and the acid generating agent (B), the composition (II) has favorable sensitivity to exposure light, and enables a resist pattern to be formed with superiority with regard to each of the LWR performance and the resolution. Although not necessarily clarified and without wishing to be bound by any theory, the reason for achieving the aforementioned effects by the composition (II) due to involving such a constitution may be presumed, for example, similar to that described in the case of the composition (I).

Each component contained in the composition (II) is described below.

(A2) Polymer

The polymer (A2) has the first structural unit (hereinafter, may be also referred to as “structural unit (II-1)”) represented by the following formula (5) and the second structural unit (hereinafter, may be also referred to as “structural unit (II-2)”) represented by the following formula (6). The polymer (A2) may also have an other structural unit aside from the structural unit (II-1) and the structural unit (II-2). The polymer (A2) may have one, or two or more types of each structural unit.

Each structural unit contained in the polymer (A2) will be described below.

Structural Unit (II-1)

The structural unit (II-1) is represented by the following formula (5). When the polymer (A2) contains the structural unit (II-1), hydrophilicity of the resist film can be increased, solubility in a developer solution can be appropriately adjusted, and further, adhesiveness of the resist pattern to the substrate can be improved. Furthermore, in a case of using an extreme ultraviolet ray or an electron beam as the radioactive ray for irradiation in a step of irradiating of the method of forming a resist pattern, as described later, the sensitivity to exposure light can be further improved.

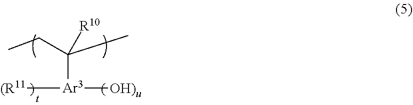

In the above formula (5), R¹⁰ represents a hydrogen atom, a fluorine atom, a methyl group, or a trifluoromethyl group; Ar³ represents a group obtained by removing (t+u+1) hydrogen atoms on an aromatic ring from an arene having 6 to 20 ring atoms; t is an integer of 0 to 10, wherein in a case in which t is 1, R¹¹ represents a halogen atom or a monovalent organic group having 1 to 20 carbon atoms, and in a case in which t is no less than 2, a plurality of R¹¹s are identical or different and each R¹¹ represents a halogen atom or a monovalent organic group having 1 to 20 carbon atoms, or at least two of the plurality of R¹¹s taken together represent a ring structure having 4 to 20 ring atoms together with the carbon chain to which the at least two of the plurality of R¹¹s bond; and u is an integer of 1 to 11, wherein a sum of (t+u) is no greater than 11.

The structural unit (II-1) corresponds to the structural unit represented by the above formula (4), which is exemplified as the structural unit (I-1) described above, in which R⁸ represents a single bond in the above formula (4). Thus, each of R¹⁰, Ar³, R¹¹, t, and u in the above formula (5) is defined similarly to each of R⁷, Ar², R⁹, r, and s, respectively, in the above formula (4).

A proportion of the structural unit (II-1) in the polymer (A2) is similar to the proportion of the structural unit (I-1) in the polymer (A1) in the composition (I), described above.

Structural Unit (II-2)

The structural unit (II-2) is represented by the following formula (6). The structural unit (II-2) includes an acid-labile group. When the polymer (A2) contains the acid-labile group in the structural unit (II-2), the acid-labile group is dissociated in light-exposed regions by an action of an acid generated from the acid generating agent (B) in the exposing, and a difference in solubility in a developer solution emerges between the light-exposed regions and the light-unexposed regions, thereby enabling forming the resist pattern. It is to be noted that in the following formula (6), a group bonding to an oxy-oxygen atom derived from the carboxy group corresponds to the acid-labile group.

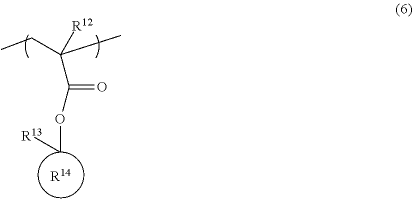

In the above formula (6), R¹² represents a hydrogen atom, a fluorine atom, a methyl group, or a trifluoromethyl group; R¹³ represents a hydrogen atom or a monovalent hydrocarbon group having 1 to 20 carbon atoms; and R¹⁴ represents a divalent alicyclic hydrocarbon group having 3 to 30 ring atoms.

R¹² and R¹³ in the above formula (6) are each defined similarly to each of R¹ and R², respectively, in the above formula (1).

Examples of the divalent alicyclic hydrocarbon group having 3 to 30 ring atoms represented by R¹⁴ include: groups obtained by removing two hydrogen atoms from one carbon atom constituting a monocyclic saturated alicyclic structure such as a cyclopentane ring or a cyclohexane ring; groups obtained by removing two hydrogen atoms from one carbon atom constituting a polycyclic saturated alicyclic structure such as a norbornane ring, an adamantane ring, a tricyclodecane ring, or a tetracyclododecane ring; groups obtained by removing two hydrogen atoms from one carbon atom constituting a monocyclic unsaturated alicyclic structure such as a cyclopentene ring or a cyclohexene ring; groups obtained by removing two hydrogen atoms from one carbon atom constituting an unsaturated alicyclic structure such as a norbornene ring, a tricyclodecene ring, or a tetracyclododecene ring; and the like.

A proportion of the structural unit (II-2) in the polymer (A2) is similar to the proportion of the structural unit (I-2) in the polymer (A1) in the composition (I), described above.

Other Structural Unit

The other structural unit which may be included in the polymer (A2) is similar to the other structural unit in the composition (I), described above.

(B) Acid Generating Agent

The acid generating agent contained in the composition (II) is similar to the acid generating agent (B) in the composition (I), described above.

(C) Acid Diffusion Controller

The acid diffusion controller (C) which may be contained in the composition (II) is similar to the acid diffusion controller (C) in the composition (I), described above.

(D) Organic Solvent

The organic solvent (D) which may be contained in the composition (II) is similar to the organic solvent (D) in the composition (I), described above.

Other Optional Component(s)

The other optional component(s) which may be contained in the composition (II) is similar to the other optional component(s) in the composition (I), described above.

### Preparation Procedure of Radiation-Sensitive Resin Composition

The radiation-sensitive resin composition (the composition (I) or the composition (II)) may be prepared, for example, by mixing the polymer (A1) or the polymer (A2) and the acid generator (B), as well as the acid diffusion controller (C), the organic solvent (D), the other optional component(s), and the like, which are added as needed, in a certain ratio, and preferably filtering a thus resulting mixture through a membrane filter having a pore size of no greater than 0.2 μm.

### Method of Forming Resist Pattern

The method of forming a resist pattern according to another embodiment of the present invention includes: a step of applying the radiation-sensitive resin composition according to the one embodiment of the invention directly or indirectly on a substrate (hereinafter, may be also referred to as “applying step”); a step of exposing a resist film formed by the applying step (hereinafter, may be also referred to as “exposing step”); and a step of developing the resist film exposed (hereinafter, may be also referred to as “developing step”).

According to the method of forming a resist film, due to using the radiation-sensitive resin composition in the applying step, formation of a resist pattern with favorable sensitivity to exposure light and superiority with regard to both of the LWR performance and the sensitivity is enabled.

Each step included in the method of forming a resist pattern will be described below.

Applying Step

In this step, the radiation-sensitive resin composition is applied directly or indirectly on a substrate. Accordingly, a resist film is formed. The substrate is exemplified by a conventionally well-known substrate such as a silicon wafer, a wafer coated with silicon dioxide or aluminum, and the like. In addition, an organic or inorganic antireflective film disclosed in, for example, Japanese Examined Patent Application, Publication No. H6-12452, Japanese Unexamined Patent Application, Publication No. S59-93448, or the like may be provided on the substrate. An application procedure is exemplified by spin-coating, cast coating, roll-coating, and the like. After the application, prebaking (PB) may be carried out as needed for evaporating the solvent remaining in the coating film. The lower limit of a PB temperature is preferably 60° C., and more preferably 80° C. The upper limit of the PB temperature is preferably 150° C., and more preferably 140° C. The lower limit of a PB time period is preferably 5 sec, and more preferably 10 sec. The upper limit of the PB time period is preferably 600 sec, and more preferably 300 sec. The lower limit of an average thickness of the resist film formed is preferably 10 nm, and more preferably 20 nm. The upper limit of the average thickness is preferably 1,000 nm, and more preferably 500 nm.

Exposing Step

In this step, the resist film formed by the applying step is exposed. This exposure is carried out by irradiation with an exposure light through a photomask (as the case may be, through a liquid immersion medium such as water). Examples of the exposure light include electromagnetic waves such as visible light rays, ultraviolet rays, far ultraviolet rays, extreme ultraviolet rays (EUV), X-rays and γ-rays; charged particle rays such as electron beams and α-rays, and the like, which may be selected in accordance with a line width and the like of the intended pattern. Of these, far ultraviolet rays, EUV or electron beams are preferred; an ArF excimer laser beam (wavelength: 193 nm), a KrF excimer laser beam (wavelength: 248 nm), EUV, or an electron beam is more preferred; an ArF excimer laser beam, EUV, or an electron beam is still more preferred, and EUV or an electron beam is particularly preferred. It is to be noted that exposure conditions such as exposure dose and the like may be appropriately selected in accordance with a formulation of the radiation-sensitive resin composition, type(s) of additive(s), the type of exposure light, and the like.

It is preferred that post exposure baking (PEB) is carried out after the exposure to promote dissociation of the acid-labile group included in the polymer (A) mediated by the acid generated from the acid generator (B), etc. upon the exposure in exposed regions of the resist film. This PEB enables an increase in a difference in solubility of the resist film in a developer solution between the light-exposed regions and light-unexposed regions. The lower limit of a PEB temperature is preferably 50° C., more preferably 80° C., and still more preferably 90° C. The upper limit of the PEB temperature is preferably 180° C., and more preferably 130° C. The lower limit of a PEB time period is preferably 5 sec, more preferably 10 sec, and still more preferably 30 sec. The upper limit of the PEB time period is preferably 600 sec, more preferably 300 sec, and still more preferably 100 sec.

Developing Step

In this step, the resist film exposed is developed. Accordingly, formation of a predetermined resist pattern is enabled. The development is typically followed by washing with a rinse agent such as water or an alcohol and then drying. The development procedure in the developing step may be carried out by either development with an alkali, or development with an organic solvent.

In the case of the development with an alkali, the developer solution for use in the development is exemplified by: alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo-[5.4.0]-7-undecene, and 1,5-diazabicyclo-[4.3.0]-5-nonene; and the like. Of these, an aqueous TMAH solution is preferred, and a 2.38% by mass aqueous TMAH solution is more preferred.

In the case of the development with an organic solvent, the developer solution is exemplified by: an organic solvent such as a hydrocarbon solvent, an ether solvent, an ester solvent, a ketone solvent, and an alcohol solvent; a solution containing the organic solvent; and the like. An exemplary organic solvent includes one, or two or more types of the solvents exemplified as the organic solvent (D) in the radiation-sensitive resin composition of the one embodiment of the present invention, and the like. Of these, the ester solvent or the ketone solvent is preferred. The ester solvent is preferably an acetic acid ester solvent, and more preferably n-butyl acetate. The ketone solvent is preferably the chain ketone, and more preferably 2-heptanone. The lower limit of a content of the organic solvent in the developer solution is preferably 80% by mass, more preferably 90% by mass, still more preferably 95% by mass, and particularly preferably 99% by mass. Components other than the organic solvent in the developer solution are exemplified by water, silicone oil, and the like.

Examples of the development procedure include: a dipping procedure in which the substrate is immersed for a given time period in the developer solution charged in a container; a puddle procedure in which the developer solution is placed to form a dome-shaped bead by way of the surface tension on the surface of the substrate for a given time period to conduct a development; a spraying procedure in which the developer solution is sprayed onto the surface of the substrate; a dynamic dispensing procedure in which the developer solution is continuously applied onto the substrate, which is rotated at a constant speed, while scanning with a developer solution-application nozzle at a constant speed; and the like.

The resist pattern to be formed according to the method of forming a resist pattern is exemplified by a line-and-space pattern, a hole pattern, and the like.

### EXAMPLES

Hereinafter, the present invention is explained in detail by way of Examples, but the present invention is not in any way limited to these Examples. Measuring methods for various types of physical properties are shown below.

Weight Average Molecular Weight (Mw), Number Average Molecular Weight (Mn), and Dispersity Index (Mw/Mn)

Measurements of the Mw and the Mn of the polymer were carried out by gel permeation chromatography (GPC) using GPC columns available from Tosoh Corporation (“G2000 HXL”×2, “G3000 HXL”×1, and “G4000 HXL”×1) under the following conditions. Furthermore, a dispersity index (Mw/Mn) was calculated according to measurement results of the Mw and the Mn.

elution solvent: tetrahydrofuran

flow rate: 1.0 mL/min

sample concentration: 1.0% by mass

amount of injected sample: 100 uL

column temperature: 40° C.

detector: differential refractometer

standard substance: mono-dispersed polystyrene

Proportion of Each Structural Unit of Polymer

The proportion of each structural unit of each polymer was determined by ¹³C-NMR analysis using a nuclear magnetic resonance apparatus (“JNM-Delta400” available from JEOL, Ltd.).

### Synthesis of Polymer (A)

Monomers used for synthesizing each polymer in the Examples and Comparative Examples are shown below. It is to be noted that in the following Synthesis Examples, unless otherwise specified particularly, the term “parts by mass” means a value, provided that the total mass of the monomers used was 100 parts by mass, and the term “mol %” means a value, provided that the total mol number of the monomers used was 100 mol %.

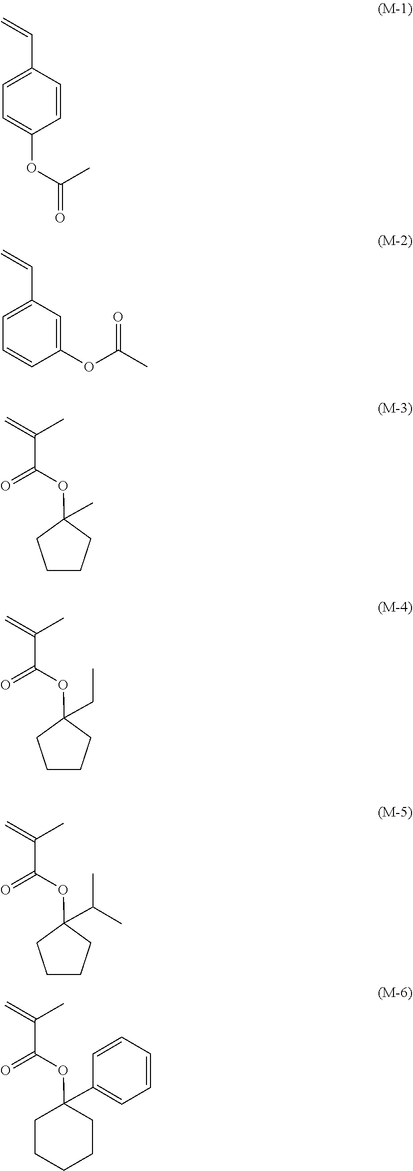

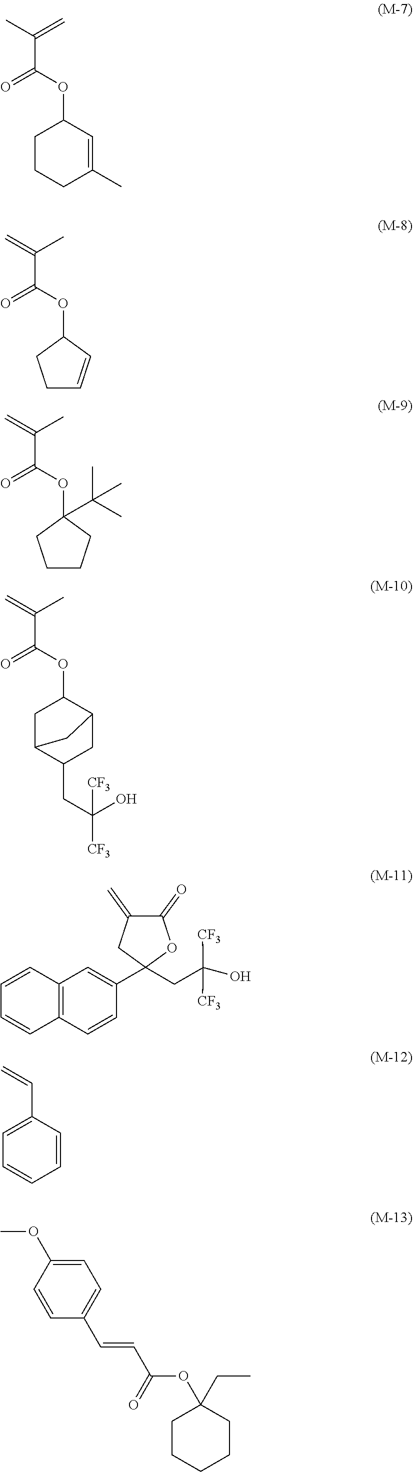

### Synthesis Example 1: Synthesis of Polymer (A-1)

The monomer (M-1) and the monomer (M-3) were dissolved in 200 parts by mass of propylene glycol-1-monomethyl ether such that the molar ratio became 40/60 (mol %). Next, a monomer solution was prepared by adding 6 mol % azobisisobutyronitrile (AIBN) as an initiator. Meanwhile, to an empty reaction vessel were charged 100 parts by mass of propylene glycol-1-monomethyl ether, which were then heated to 85° C. with stirring. Next, the monomer solution prepared as described above was added dropwise to the reaction vessel over 3 hrs, a thus resulting solution was further heated for 3 hrs at 85° C., and a polymerization reaction was allowed to proceed for 6 hrs, with the time of the start of the dropwise addition regarded as the time of the start of the polymerization reaction. After completion of the polymerization reaction, the polymerization solution was cooled to room temperature. The polymerization solution thus cooled was charged into 500 parts by mass of hexane with respect to 100 parts by mass of the polymerization solution, and a thus precipitated white powder was filtered off. The white powder obtained by the filtration was washed twice with 100 parts by mass of hexane with respect to 100 parts by mass of the polymerization solution, followed by filtering off and dissolution in 300 parts by mass of propylene glycol-l-monomethyl ether. Next, 500 parts by mass of methanol, 50 parts by mass of triethylamine, and 10 parts by mass of ultra-pure water were added to a resulting solution, and a hydrolysis reaction was performed at 70° C. for 6 hrs with stirring. After completion of the hydrolysis reaction, the remaining solvent was distilled away and a solid thus obtained was dissolved in 100 parts by mass of acetone. The solution was added dropwise to 500 parts by mass of water to permit coagulation of the polymer, and a solid thus obtained was filtered off. Drying at 50° C. for 12 hrs gave a white powdery polymer (A-1). The Mw of the polymer (A-1) was 5,700, and the Mw/Mn was 1.61. Furthermore, as a result of the ¹³C-NMR analysis, the proportions of the structural units derived from (M-1) and (M-3) were, respectively, 41.2 mol % and 58.8 mol %.

### Synthesis Examples 2 to 9: Synthesis of Polymer (A-2) to Polymer (A-9)

Polymers (A-2) to (A-9) were synthesized by a similar operation to that of Synthesis Example 1 except that monomers of the type and in the proportion shown in Table 1 below were used. The proportion and the physical property values (the Mw and the Mw/Mn) of each structural unit of each polymer thus obtained are shown together in Table 1. It is to be noted that in Table 1, “-” indicates that the corresponding monomer was not used.

### Synthesis Example 10: Synthesis of Polymer (A-10)

Using monomers of the types and in the proportions shown in Table 1 below, polymer (A-10) was synthesized in accordance with the synthesis procedure of “resin (4)” described in Japanese Unexamined Patent Publication, Publication No. 2007-206638. The proportion and the physical property values (the Mw and the Mw/Mn) of each structural unit of the polymers thus obtained are shown together in Table 1.

TABLE 1

| | | Monomer that gives first | | | Monomer that gives | | | Monomer that gives | | | | |
|---|---|---|---|---|---|---|---|---|---|---|---|---|
| | | structural unit | | | second structural unit | | | other structural unit | | | | |
| | | | | proportion of | | | proportion of | | | proportion of | Physical | |
| | (A) | | proportion | structural unit | | proportion | structural unit | | proportion | structural unit | property values | |
| — | Polymer | type | (mol %) | (mol %) | type | (mol %) | (mol %) | type | (mol %) | (mol %) | Mw | Mw/Mn |
| Synthesis | A-1 | M-1^{a} | 40 | 41.2 | M-3 | 60 | 58.8 | — | — | — | 5700 | 1.61 |
| Example 1 | | | | | | | | | | | | |
| Synthesis | A-2 | M-1^{a} | 40 | 42.3 | M-4 | 60 | 57.7 | — | — | — | 5800 | 1.64 |
| Example 2 | | | | | | | | | | | | |
| Synthesis | A-3 | M-1^{a} | 30 | 33.1 | M-5 | 60 | 56.8 | — | — | — | 6100 | 1.65 |
| Example 3 | | | | | | | | | | | | |
| | | M-1^{a} | 10 | 10.1 | | | | — | — | — | | |
| Synthesis | A-4 | M-1^{a} | 40 | 41.9 | M-6 | 60 | 58.1 | — | — | — | 6200 | 1.50 |
| Example 4 | | | | | | | | | | | | |
| Synthesis | A-5 | M-1^{a} | 40 | 39.9 | M-7 | 60 | 60.1 | — | — | — | 5500 | 1.54 |
| Example 5 | | | | | | | | | | | | |
| Synthesis | A-6 | M-1^{a} | 40 | 40.1 | M-8 | 60 | 59.9 | — | — | — | 5400 | 1.53 |
| Example 6 | | | | | | | | | | | | |
| Synthesis | A-7 | M-1^{a} | 40 | 43.2 | M-9 | 60 | 56.8 | — | — | — | 6000 | 1.67 |
| Example 7 | | | | | | | | | | | | |
| Synthesis | A-8 | M-1^{a} | 30 | 30.4 | M-3 | 60 | 58.2 | M-10 | 10 | 11.4 | 6900 | 1.70 |
| Example 8 | | | | | | | | | | | | |
| Synthesis | A-9 | M-1^{a} | 30 | 30.2 | M-3 | 60 | 59.1 | M-11 | 10 | 10.7 | 6800 | 1.65 |
| Example 9 | | | | | | | | | | | | |
| Synthesis | A-10 | M-1^{a} | 55 | 54.3 | — | — | — | M-12 | 20 | 19.2 | 8000 | 1.62 |
| Example 10 | | | | | | | | | | | | |
| | | | | | | | | M-13 | 25 | 26.5 | | |

^{a}present as hydroxystyrene

### Synthesis of Acid Generating Agent (B)

### Synthesis Example 11: Synthesis of Acid Generating Agent (B-1)

Into a reaction vessel were charged 40.3 mmol of diphenylsulfoxide and 290 g of tetrahydrofuran. After stirring a resulting mixture at 0° C., 121 mmol of chlorotrimethylsilane (TMS-Cl) was added thereto by dropwise addition, followed by dropwise addition of 121 mmol of 2-naphthylmagnesium bromide. After stirring a resulting mixture for 1 hour at room temperature, a 2 M aqueous hydrochloric acid solution was added, and then an aqueous layer was separated. The aqueous layer thus obtained was washed with diethyl ether, and an organic layer was extracted with dichloromethane. After drying over sodium sulfate, the solvent was distilled away, and then, purifying by column chromatography gave a compound (hereinafter, may be also referred to as “bromide salt (S-1)”) represented by the following formula (S-1).

Next, into a reaction vessel were charged 20.0 mmol of the bromide salt (S-1) obtained as described above, 20.0 mmol of a compound (hereinafter, may be also referred to as “ammonium salt (P-1)”) represented by the following formula (P-1), 150 g of dichloromethane, and 150 g of ultra-pure water. After stirring a resulting mixture for 2 hrs at room temperature, an organic layer was separated. The organic layer thus obtained was washed with ultra-pure water. After drying over sodium sulfate, the solvent was distilled away, and then, purifying by column chromatography gave a compound (hereinafter, may be also referred to as “acid generating agent (B-1)”) represented by the following formula (B-1). A synthesis scheme of the acid generating agent (B-1) is shown below.

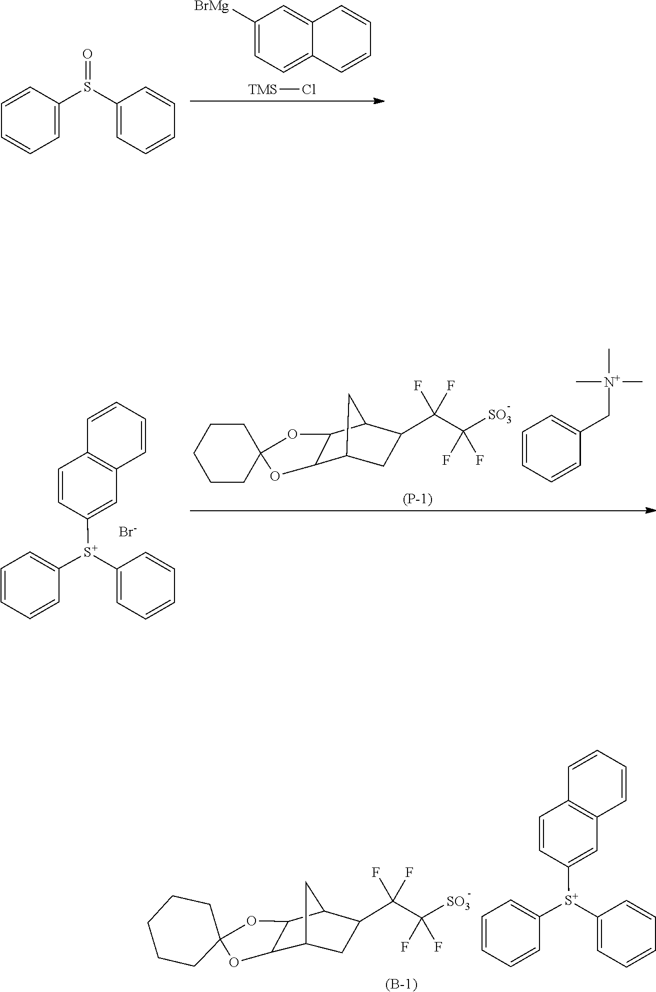

### Synthesis Examples 12 to 24: Synthesis of Acid Generating Agents (B-2) to (B-14)

Compounds (hereinafter, may be also referred to as “acid generating agents (B-2) to (B-14)”) represented by the following formulae (B-2) to (B-14) were synthesized by a similar operation to that of Synthesis Example 11 except that each precursor was selected as appropriate.

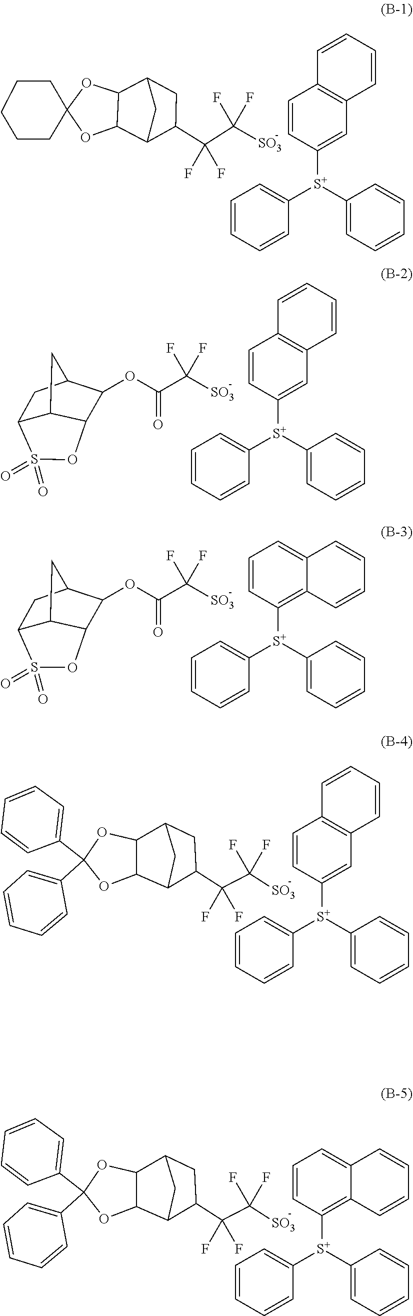

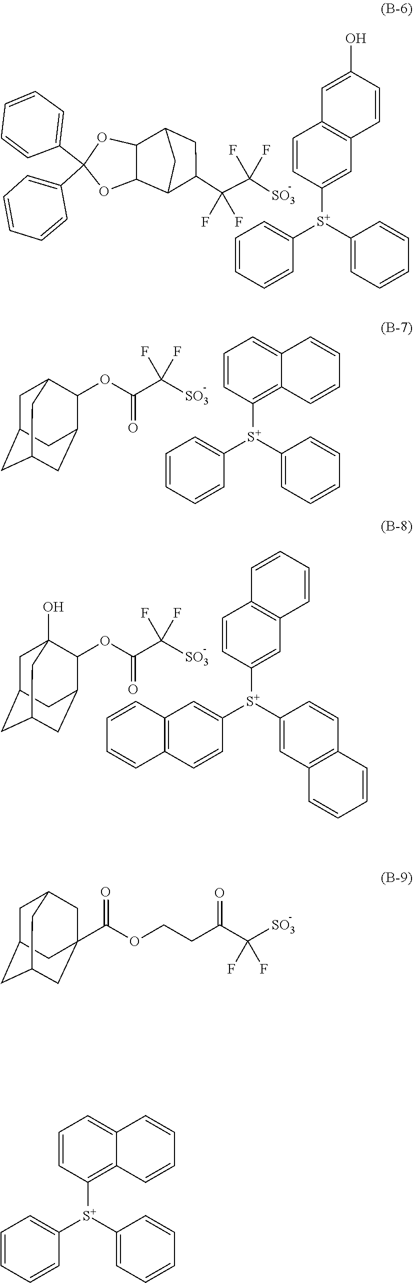

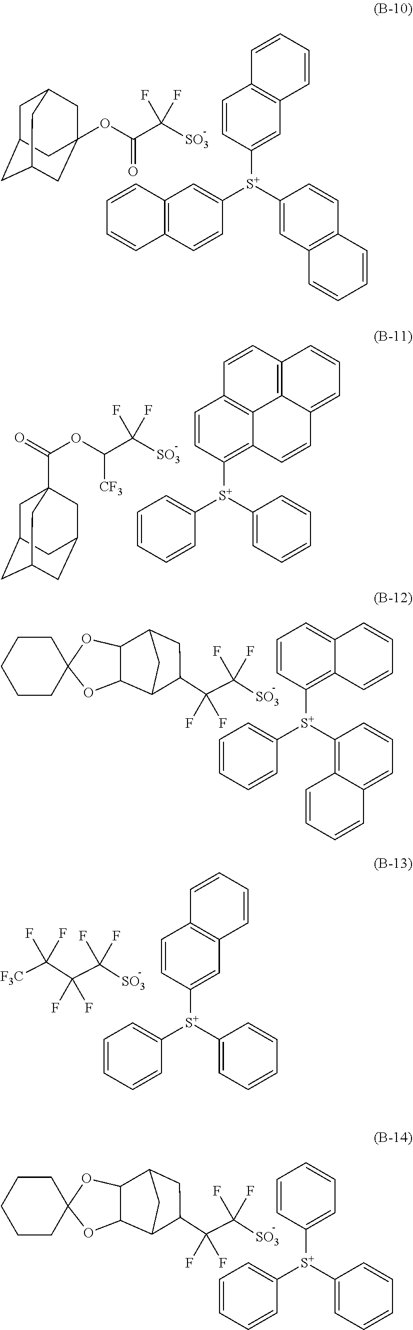

### Preparation of Radiation-Sensitive Resin Composition

Components other than the polymer (A) and the acid generating agent (B) used in preparing each radiation-sensitive resin composition are shown below. It is to be noted that in the following Examples and Comparative Examples, unless otherwise specified particularly, the term “parts by mass” means a value, provided that the mass of the polymer used was 100 parts by mass, and the term “mol %” means a value, provided that the mol number of the acid generating agent (B) used was 100 mol %.

(C) Acid Diffusion Control Agent

(C-1) and (C-2): compounds represented by the following formulae (C-1) and (C-2)

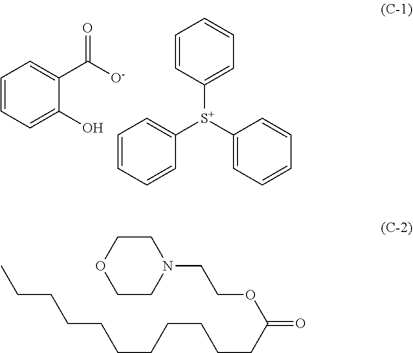 [SMILES: O=C([O-])c1ccccc1O ; c1ccc([S+](c2ccccc2)c2ccccc2)cc1 ; CCCCCCCCCCCC(=O)OCCN1CCOCC1]

(D) Organic Solvent

D-1: propylene glycol monomethyl ether acetate

D-2: propylene glycol 1-monomethyl ether

### Example 1: Preparation of Radiation-Sensitive Resin Composition (R-1)

A radiation-sensitive resin composition (R-1) was prepared by: mixing 100 parts by mass of (A-1) as the polymer (A), 20 parts by mass of (B-1) as the acid generating agent (B), 20 mol % of (C-1) as the acid diffusion control agent (C), and 4,800 parts by mass of (D-1) and 2,000 parts by mass of (D-2) as the organic solvent (D), and filtering a resulting mixture through a membrane filter having a pore size of 0.2 μm.

### Examples 2 to 17 and Comparative Examples 1 to 2

Radiation-sensitive resin compositions (R-2) to (R-17) and (CR-1) to (CR-2) were prepared in a similar manner to Example 1, except that for each component, the type and content shown in Table 2 below were used.

TABLE 2

| | | | | (B) Acid | | | | | |
|---|---|---|---|---|---|---|---|---|---|
| | Radiation- | (A) Polymer | | generating agent | | (C) Acid diffusion | | (D) Organic solvent | |
| | sensitive | | content | | content | control agent | | | content |
| | resin | | (parts | | (parts | | proportion | | (parts |
| — | composition | type | by mass) | type | by mass) | type | (mol %) | type | by mass) |
| Example 1 | R-1 | A-1 | 100 | B-1 | 20 | C-1 | 20 | D-1/D-2 | 4,800/2,000 |
| Example 2 | R-2 | A-1 | 100 | B-2 | 20 | C-1 | 20 | D-1/D-2 | 4,800/2,000 |
| Example 3 | R-3 | A-1 | 100 | B-3 | 20 | C-1 | 20 | D-1/D-2 | 4,800/2,000 |
| Example 4 | R-4 | A-1 | 100 | B-4 | 20 | C-1 | 20 | D-1/D-2 | 4,800/2,000 |
| Example 5 | R-5 | A-1 | 100 | B-5 | 20 | C-1 | 20 | D-1/D-2 | 4,800/2,000 |
| Example 6 | R-6 | A-1 | 100 | B-6 | 20 | C-1 | 20 | D-1/D-2 | 4,800/2,000 |
| Example 7 | R-7 | A-1 | 100 | B-7 | 20 | C-1 | 20 | D-1/D-2 | 4,800/2,000 |
| Example 8 | R-8 | A-1 | 100 | B-10 | 20 | C-1 | 20 | D-1/D-2 | 4,800/2,000 |
| Example 9 | R-9 | A-1 | 100 | B-12 | 20 | C-1 | 20 | D-1/D-2 | 4,800/2,000 |
| Example 10 | R-10 | A-2 | 100 | B-1 | 20 | C-1 | 20 | D-1/D-2 | 4,800/2,000 |
| Example 11 | R-11 | A-3 | 100 | B-1 | 20 | C-1 | 20 | D-1/D-2 | 4,800/2,000 |
| Example 12 | R-12 | A-4 | 100 | B-1 | 20 | C-1 | 20 | D-1/D-2 | 4,800/2,000 |
| Example 13 | R-13 | A-5 | 100 | B-8 | 20 | C-1 | 20 | D-1/D-2 | 4,800/2,000 |
| Example 14 | R-14 | A-6 | 100 | B-9 | 20 | C-1 | 20 | D-1/D-2 | 4,800/2,000 |
| Example 15 | R-15 | A-7 | 100 | B-11 | 20 | C-2 | 20 | D-1/D-2 | 4,800/2,000 |
| Example 16 | R-16 | A-8 | 100 | B-1 | 20 | C-1 | 20 | D-1/D-2 | 4,800/2,000 |
| Example 17 | R-17 | A-9 | 100 | B-1 | 20 | C-1 | 20 | D-1/D-2 | 4,800/2,000 |
| Comparative | CR-1 | A-10 | 100 | B-13 | 20 | C-1 | 20 | D-1/D-2 | 4,800/2,000 |
| Example 1 | | | | | | | | | |
| Comparative | CR-2 | A-1 | 100 | B-14 | 20 | C-1 | 20 | D-1/D-2 | 4,800/2,000 |
| Example 2 | | | | | | | | | |

### Resist Pattern Formation (EUV Exposure, Alkali Development)

Using a spin coater (“CLEAN TRACK ACT12,” available from Tokyo Electron Limited), the radiation-sensitive resin compositions prepared as described above were each applied on an underlayer film (“AL412,” available from Brewer Science, Inc.) formed on a 12-inch silicon wafer, the underlayer film having an average thickness of 20 nm being provided thereon, and prebaking (PB) was conducted at 130° C. for 60 sec. Thereafter, by cooling at 23° C. for 30 sec, a resist film having an average thickness of 50 nm was formed. Next, the resist film was exposed using an EUV scanner (model “NXE3300,” available from ASML Co.) with NA of 0.33 under an illumination condition of Conventional s=0.89, and with a mask of imecDEFECT32FFR02, and then subjected to PEB at 130° C. for 60 sec. Thereafter, the resist film was developed at 23° C. for 30 sec by using a 2.38% by mass aqueous TMAH solution as an alkaline developer solution to form a positive-tone resist pattern (32 nm line-and-space pattern).

### Evaluations

With regard to the resist patterns formed as described above, each radiation-sensitive resin composition was evaluated on the sensitivity, the LWR performance, and the resolution thereof in accordance with the following methods. A scanning electron microscope (“CG-4100,” available from Hitachi High-Technologies Corporation) was used for line-width measurement of the resist patterns. The results of the evaluations are shown in Table 3 below.

Sensitivity

An exposure dose at which a 32-nm line-and-space pattern was formed in the aforementioned resist pattern formation was defined as an optimum exposure dose, and this optimum exposure dose was adopted as sensitivity (mJ/cm²). The sensitivity was evaluated to be: “favorable” in a case of being no greater than 30 mJ/cm²; and “unfavorable” in a case of being greater than 30 mJ/cm².

LWR Performance

The resist patterns formed as described above were observed from above using the scanning electron microscope. Line widths were measured at 50 arbitrary sites, and then a 3 Sigma value was determined from distribution of the measurements and defined as “LWR” (nm). The value being smaller reveals less line roughness, indicating better LWR performance. The LWR performance was evaluated to be: “favorable” in a case of the LWR being no greater than 4.0 nm; and “unfavorable” in a case of the LWR being greater than 4.0 nm.

Resolution

A dimension of a minimum resist pattern being resolved at the optimum exposure dose was measured when the mask pattern size for forming the line-and-space (1L/1S) was changed, and the measurement value was defined as “resolution” (nm). The value being smaller enables formation of a finer pattern, indicating a more favorable resolution. The resolution was evaluated to be: “favorable” in a case being no greater than 25 nm; and “unfavorable” in a case of being greater than 25 nm.

TABLE 3

| | Radiation- | | | |
|---|---|---|---|---|
| | sensitive resin | Sensitivity | LWR | Resolution |
| — | composition | (mJ/cm²) | (nm) | (nm) |
| Example 1 | R-1 | 26 | 3.6 | 23 |
| Example 2 | R-2 | 26 | 3.4 | 21 |
| Example 3 | R-3 | 26 | 3.5 | 22 |
| Example 4 | R-4 | 27 | 3.3 | 21 |
| Example 5 | R-5 | 28 | 3.4 | 23 |
| Example 6 | R-6 | 27 | 3.6 | 22 |
| Example 7 | R-7 | 26 | 3.6 | 22 |
| Example 8 | R-8 | 29 | 3.6 | 22 |
| Example 9 | R-9 | 28 | 3.6 | 23 |
| Example 10 | R-10 | 27 | 3.5 | 22 |
| Example 11 | R-11 | 26 | 3.5 | 22 |
| Example 12 | R-12 | 28 | 3.5 | 23 |
| Example 13 | R-13 | 25 | 3.5 | 22 |
| Example 14 | R-14 | 25 | 3.5 | 23 |
| Example 15 | R-15 | 27 | 3.5 | 24 |
| Example 16 | R-16 | 26 | 3.4 | 22 |
| Example 17 | R-17 | 26 | 3.5 | 22 |
| Comparative | CR-1 | 33 | 4.4 | 29 |
| Example 1 | | | | |
| Comparative | CR-2 | 32 | 4.1 | 26 |
| Example 2 | | | | |

As is clear from the results shown in Table 3, with regard to the radiation-sensitive resin compositions of the Examples, the sensitivity, the LWR performance, and the resolution were favorable when compared to those of the radiation-sensitive resin compositions of the Comparative Examples.

The radiation-sensitive resin composition and the method of forming a resist pattern of the embodiments of the present invention enable a resist pattern to be formed with favorable sensitivity to exposure light and superiority with regard to each of the LWR performance and the resolution. Therefore, these can be suitably used in manufacturing processes of semiconductor devices, in which further progress of miniaturization is expected in the future.

Obviously, numerous modifications and variations of the present invention are possible in light of the above teachings. It is therefore to be understood that within the scope of the appended claims, the invention may be practiced otherwise than as specifically described herein.

What is claimed is:

1. A radiation-sensitive resin composition comprising:
a polymer which comprises: a first structural unit comprising a phenolic hydroxyl group; and a second structural unit represented by formula (1); and
a radiation-sensitive acid generating agent which comprises a compound represented by formula (2): 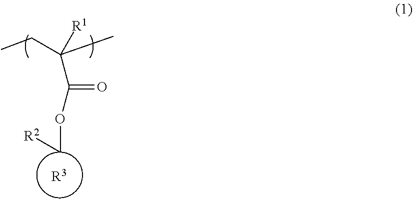
wherein, in the formula (1), R¹ represents a hydrogen atom, a fluorine atom, a methyl group, or a trifluoromethyl group; R² represents a hydrogen atom or a monovalent hydrocarbon group having 1 to 20 carbon atoms; and R³ represents a divalent monocyclic alicyclic hydrocarbon group having 3 to 12 ring atoms, and 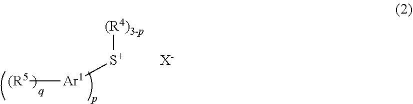
in the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two of the plurality of R⁵s taken together represent an alicyclic structure having 4 to 20 ring atoms or an aliphatic heterocyclic structure having 4 to 20 ring atoms, together with the carbon chain to which the at least two of the plurality of R⁵s bond; in a case in which p is no less than 2, a plurality of Ar¹s are identical or different from each other, and a plurality of q's are identical or different from each other; and X⁻ represents a monovalent anion.

2. The radiation-sensitive resin composition according to claim 1, wherein p in the formula (2) is 1.

3. The radiation-sensitive resin composition according to claim 1, wherein X⁻ in the formula (2) is represented by formula (3):
R⁶—Y⁻ (3)
wherein, in the formula (3), R⁶ represents a monovalent organic group having 1 to 30 carbon atoms; and Y⁻ represents a group obtained by removing one proton from an acid group.

4. The radiation-sensitive resin composition according to claim 3, wherein in the formula (2), in a case in which q is 1, R⁵ represents a halogen atom, a nitro group, a monovalent unsubstituted hydrocarbon group having 1 to 20 carbon atoms, or a monovalent hydrocarbon group having 1 to 20 carbon atoms in which a part or all of hydrogen atoms are substituted with a substituent, and in a case in which q is no less than 2, a plurality of R⁵s are identical or different and each R⁵ represents a halogen atom, a nitro group, a monovalent unsubstituted hydrocarbon group having 1 to 20 carbon atoms, or a monovalent hydrocarbon group having 1 to 20 carbon atoms in which a part or all of hydrogen atoms are substituted with a substituent, or at least two of the plurality of R⁵s taken together represent an alicyclic structure having 4 to 20 ring atoms or an aliphatic heterocyclic structure having 4 to 20 ring atoms, together with the carbon chain to which the at least two of the plurality of R⁵s bond.

5. The radiation-sensitive resin composition according to claim 4, wherein the acid group is a sulfo group or a carboxy group.

6. The radiation-sensitive resin composition according to claim 1, wherein the first structural unit is represented by formula (4): 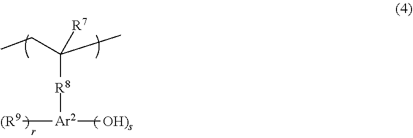
wherein, in the formula (4), R⁷ represents a hydrogen atom, a fluorine atom, a methyl group, or a trifluoromethyl group; R⁸ represents a single bond, −O—, —COO—, or —CONH—; Ar² represents a group obtained by removing (r+s+1) hydrogen atoms on an aromatic ring from an arene having 6 to 20 ring atoms; r is an integer of 0 to 10, wherein in a case in which r is 1, R⁹ represents a halogen atom or a monovalent organic group having 1 to 20 carbon atoms, and in a case in which r is no less than 2, a plurality of R⁹s are identical or different and each R⁹ represents a halogen atom or a monovalent organic group having 1 to 20 carbon atoms, or at least two of the plurality of R⁹s taken together represent a ring structure having 4 to 20 ring atoms together with the carbon chain to which the at least two of the plurality of R⁹s bond; and s is an integer of 1 to 11, wherein a sum of r and s is no greater than 11.

7. A radiation-sensitive resin composition comprising:
a polymer which comprises: a first structural unit represented by formula (5); and a second structural unit represented by formula (6); and
a radiation-sensitive acid generating agent which comprises a compound represented by formula (2): 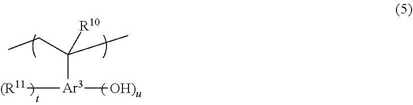
wherein, in the formula (5), R¹⁰ represents a hydrogen atom, a fluorine atom, a methyl group, or a trifluoromethyl group; Ar³ represents a group obtained by removing (t+u+1) hydrogen atoms on an aromatic ring from an arene having 6 to 20 ring atoms; t is an integer of 0 to 10, wherein in a case in which t is 1, R¹¹ represents a halogen atom or a monovalent organic group having 1 to 20 carbon atoms, and in a case in which t is no less than 2, a plurality of R¹¹s are identical or different and each R¹¹ represents a halogen atom or a monovalent organic group having 1 to 20 carbon atoms, or at least two of the plurality of R¹¹s taken together represent a ring structure having 4 to 20 ring atoms together with the carbon chain to which the at least two of the plurality of R¹¹s bond; and u is an integer of 1 to 11, wherein a sum of t and u is no greater than 11, 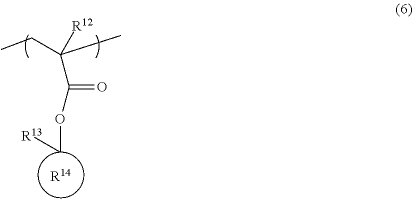
in the formula (6), R¹² represents a hydrogen atom, a fluorine atom, a methyl group, or a trifluoromethyl group; R¹³ represents a hydrogen atom or a monovalent hydrocarbon group having 1 to 20 carbon atoms; and R¹⁴ represents a divalent alicyclic hydrocarbon group having 3 to 30 ring atoms, and 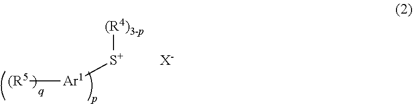
in the formula (2), Ar¹ represents a group obtained by removing (q+1) hydrogen atoms on an aromatic ring from an arene formed by condensation of at least two benzene rings; R⁴ represents a monovalent organic group having 1 to 20 carbon atoms; p is an integer of 1 to 3, wherein in a case in which p is 1, two R⁴s are identical or different from each other; q is an integer of 0 to 7, wherein in a case in which q is 1, R⁵ represents a halogen atom, a hydroxy group, a nitro group, or a monovalent organic group having 1 to 20 carbon atoms, and in a case in which q is no less than 2, a plurality of R⁵s are identical or different and each R⁵ represents a halogen atom, a hydroxy group, a nitro group, or a monovalent organic group having 1 to 20 carbon atoms, or at least two R⁵s taken together represent an alicyclic structure having 4 to 20 ring atoms or an aliphatic heterocyclic structure having 4 to 20 ring atoms, together with the carbon chain to which the at least two R⁵s bond; in a case in which p is no less than 2, a plurality of Ar¹s are identical or different from each other, and a plurality of q's are identical or different from each other; and X⁻ represents a monovalent anion.

8. The radiation-sensitive resin composition according to claim 7, wherein p in the formula (2) is 1.

9. The radiation-sensitive resin composition according to claim 7, wherein X⁻ in the formula (2) is represented by formula (3):
R⁶—Y⁻ (3)
wherein, in the formula (3), R⁶ represents a monovalent organic group having 1 to 30 carbon atoms; and Y⁻ represents a group obtained by removing one proton from an acid group.

10. The radiation-sensitive resin composition according to claim 9, wherein in the formula (2), in a case in which q is 1, R⁵ represents a halogen atom, a nitro group, a monovalent unsubstituted hydrocarbon group having 1 to 20 carbon atoms, or a monovalent hydrocarbon group having 1 to 20 carbon atoms in which a part or all of hydrogen atoms are substituted with a substituent, and in a case in which q is no less than 2, a plurality of R⁵s are identical or different and each R⁵ represents a halogen atom, a nitro group, a monovalent unsubstituted hydrocarbon group having 1 to 20 carbon atoms, or a monovalent hydrocarbon group having 1 to 20 carbon atoms in which a part or all of hydrogen atoms are substituted with a substituent, or at least two of the plurality of R⁵s taken together represent an alicyclic structure having 4 to 20 ring atoms or an aliphatic heterocyclic structure having 4 to 20 ring atoms, together with the carbon chain to which the at least two of the plurality of R⁵s bond.

11. The radiation-sensitive resin composition according to claim 10, wherein the acid group is a sulfo group or a carboxy group.

12. The radiation-sensitive resin composition according to claim 1, which is suitable for exposure to extreme ultraviolet light or exposure to an electron beam.

13. The radiation-sensitive resin composition according to claim 7, which is suitable for exposure to extreme ultraviolet light or exposure to an electron beam.

14. A method of forming a resist pattern, the method comprising:
forming a resist film directly or indirectly on a substrate by applying the radiation-sensitive resin composition according to claim 1;
exposing the resist film; and
developing the resist film exposed.

15. A method of forming a resist pattern, the method comprising:
forming a resist film directly or indirectly on a substrate by applying the radiation-sensitive resin composition according to claim 7;
exposing the resist film; and
developing the resist film exposed.
